(19) United States
(12) Patent Application Publication
(10) Pub. No.: US 2021/0169913 A1
(43) Pub. Date: Jun. 10, 2021

(54) FCRL6 AND ITS USES RELATED TO CANCER

(71) Applicants: THE UAB RESEARCH FOUNDATION, Birmingham, AL (US); Vanderbilt University, Nashville, TN (US)

(72) Inventors: Randall S. Davis, Birmingham, AL (US); Daniel M. Schreeder, Huntsville, AL (US); Douglas B. Johnson, Nashville, TN (US); Justin M. Balko, Nashville, TN (US)

(21) Appl. No.: 16/762,682

(22) PCT Filed: Nov. 9, 2018
(86) PCT No.: PCT/US2018/060065

§ 371 Date: May 8, 2020

Related U.S. Application Data
(60) Provisional application No. 62/584,458, filed on Nov. 10, 2017.

(51) Int. Cl. A61K 31/7088; A61K 45/06; C07K 16/28

(52) U.S. Cl. CPC A61K 31/7088; A61K 2039/505; C07K 16/2818; A61K 45/06

(57) ABSTRACT

Provided herein are methods for treating and preventing PD-1-mediated resistant cancers.

This application claims the benefit of U.S. Provisional Application No. 62/584,458, filed Nov. 10, 2017, which is hereby incorporated in its entirety by this reference.

### STATEMENT REGARDING FEDERALLY FUNDED RESEARCH

This invention was made with government support under Grant No. AI067467 awarded by the National Institutes of Health. The government has certain rights in the invention.

### BACKGROUND

Immune checkpoint inhibitors that block the interaction between programmed cell death protein 1 (PD-1) and its ligand, programmed cell death protein ligand 1 (PDL-1), have transformed the treatment landscape of numerous solid tumors. These agents unleash restrained pre-existing anti-tumor immune responses, leading to durable disease control in a substantial fraction of treated patients. Despite these advances, intrinsic and acquired resistance curtails clinical benefits in most patients.

### SUMMARY

Provided herein are methods of reducing resistance to an anti-programmed cell death protein 1 (PD-1) or an anti-programmed cell death protein ligand 1 (PDL-1) therapy in a subject with cancer. The methods comprise administering to the subject with cancer an effective amount of an Fc receptor like 6 (FCRL6) inhibitor, wherein the subject has been treated with an anti-PD1 therapy or an anti-PDL-1 therapy.

Also provided are methods of treating or preventing a PD-1 mediated resistant cancer in a subject. The methods comprise administering an effective amount of FCRL6 inhibitor to a subject that has been treated with an anti-PD1 therapy or an anti-PDL-1 therapy.

Further provided are methods of diagnosing and treating a PD-1-mediated resistant cancer in a subject that has been treated with an anti-PD1 therapy or an anti-PDL-1 therapy. The methods comprise a) obtaining a biological sample from a subject that has been treated with an anti-PD1 therapy or an anti-PDL-1 therapy; b) detecting the level of FCRL6 in the sample; c) diagnosing the subject with a PD-1 mediated resistant cancer when the level of FCRL6 in the sample is increased as compared to a control sample; and d) administering an effective amount of an FCRL6 inhibitor. Alternatively, the methods comprise a) obtaining a biological sample from a subject that has been treated with an anti-PD1 therapy or an anti-PDL-1 therapy; b) detecting the level of FCRL6 and the level of LAG3 in the sample; c) diagnosing the subject with a PD-1 mediated resistant cancer when the level of FCRL6 and the level of LAG3 in the sample are increased as compared to a control sample; d) administering an effective amount of an anti-LAG3 therapy and/or an FCRL6 inhibitor to the subject.

### BRIEF DESCRIPTION OF THE FIGURES

FIGS. 1A-1I show that MHC-II/HLA-DR expression in patient tumor samples is associated with unique patterns of inflammation and enhanced CD4, CD8, and LAG-3+ infiltrate. A) Representative images of immunohistochemistry from HLA-DR+ and HLA-DR− tumors. HLA-DR is stained (DAB) and Sox10, a nuclear melanoma marker, is stained (Mach Red). B) Gene Set Analysis from RNAseq analysis of IHC-defined tumor HLA-DR+(>5% tumor cells) or HLA-DR− (<5% tumor cells) melanoma and lung specimens. After significant (FDR<10%) gene set scores were defined, scores were created as the mean of all genes in each signature for each sample and plotted as row-standardized Z-scores with heatmap representation. C) mRNA expression levels of selected genes by HLA-DR status. *p<0.05, two-tailed t test. Error bars represent the mean SEM. D) Pearson's correlation matrix of gene expression associations between immune response and inhibitory markers among PD-1 treated patient samples from melanoma and lung cancer (n=50). Data represent correlation among TPM RNAseq values, expect ‘HLA-DR_TUMOR’, which is the correlation with tumor HLA-DR percent positivity by IHC (n=41/50 available data points). Values in the individual boxes represent the Pearson's correlation coefficient. E) RNAseq expression levels of checkpoint and checkpoint ligands by HLA-DR status of the tumor. *p<0.05; **p<0.01 by two-tailed t test. F) RNAseq expression levels of checkpoint and checkpoint ligands by patient immune-related response criteria (irRC); PD: progressive disease, SD/MR: stable disease or mixed response, PR: partial response, CR: complete response, RELAPSE: sample collected at relapse/progression after initial PR/CR; *p<0.05, Tukey's post-hoc test. G) RNAseq expression levels of checkpoints in 3 pairs of matched pre-response and post-relapse specimens. P-value represents paired two-tailed t test. H) Representative IHC for LAG-3 in a melanoma sample before anti-PD-1 response and at progression. I) IHC analysis for LAG-3+ TILs in 5 paired melanoma specimens before anti-PD-1 response and at progression.

FIGS. 2A-B show interferon-gamma gene expression signatures by outcome in PD-1 treated patients. RNAseq expression levels of IFN-γ (A) and IFN-γ-responsive (B) gene signatures by melanoma and lung patient irRC are shown. *p<0.05, Tukey's post-hoc test.

FIGS. 3A-B show MHC-I and MHC-II gene expression signatures by outcome in PD-1 treated patients. RNAseq expression levels of HLA-A (A) and HLA-DRA (B) genes by melanoma and lung patient irRC are shown.

FIGS. 4A-J show that MHC-II+ breast tumors recruit CD4+ T cells and are associated with Lag-3+ TILs. A) Representative positive control for Lag-3 IHC in human tonsil. B) Representative Lag-3+ breast cancer TILs by IHC. C) Representative AQUA immunofluorescence image of a HLA-DR+ breast tumor case. D) Difference in tumor-specific HLA-DR expression among LAG-3+ and LAG-3− infiltrated triple-negative breast cancers in all patients (n=86; left) and only those patients with a % stromal TILs>20% (n=31; right). ***p<0.001, two-tailed t test. E) CD4 and CD8 infiltration in HLA-DR-high (HI) and HLA-DR-low (LO) expressing tumors. **p<0.01, two-tailed t test. F) Association of stromal (non-cytokeratin+) PDL-1 expression with HLA-DR expression. **p<0.01; two-tailed t test. G) Flow cytometry of MMTV-neu cells transduced with Ciita or vector control, stained with anti-MHC—II-Alexa488 (IA-IE) or isotype control. H) Tumor growth or orthotopic MMTV-neu cells in wildtype syngeneic FVB/n mice. Red lines represent rejected engraftments. I) Proportions of rejected tumor engraftments for control or Ciita+ MMTV-neu cells. P-value represents fisher's exact test. J) Tumors from (H) that were not rejected were examined by H&E for TILs and T cell compartments by IHC (CD8, CD4, and Foxp3) and scored as % total TILs. P-values represent 2-tailed Mann-Whitney test.

FIGS. 5A-B show correlates of LAG-3+ TILs in TNBC. A) Percent of tumor infiltrating lymphocytes across TNBC patients, stratified by presence of LAG-3+ cells. *** p<0.001; Mann Whitney U test. B) Percent of PD-L1+ stroma (AQUA score) across TNBC patients, stratified by presence of LAG-3+ cells. *p<0.05; Mann Whitney U test.

FIG. 6 shows that there is no change in Ciita+ MMTV-neu tumor growth rate after immunologic escape. Tumor growth rates in MMTV-neu Ciita+ and puro-control tumors. Only mice which formed tumors (did not reject) are included.

FIGS. 7A-C show that enforced MHC-II expression in B16 tumors results in enhanced CD4+ TIL population. B16 tumors with enforced expression of Ciita or puro-control were harvested at ˜1 cm3 and dissociated for flow cytometry. A) MHC-II expression in CD45− (tumor) cells B) CD4+ T cells expressed as a percent of CD3 infiltrate. P value represents two-sample t test. C) CD8+ T cells expressed as a percent of CD3 infiltrate.

FIGS. 8A-F show enhanced T cell-recruiting chemokines in MHC-II+ mouse and human tumors. A) Heatmap visualization of altered gene expression levels in non-rejected tumors from FIG. 4H. Transcript counts (NanoString PanCancer Immune profiling) were row normalized/standardized for visualization. B) Gene expression levels of immune checkpoints Pd-1, Tim-3, and Lag-3 in Ciita+ and control tumors. C) NanoString gene expression data from MMTV-neu Ciita+ or vector control tumors harvested at humane endpoints (1.5-2 cm³) were queried for expression levels of T cell-attracting chemokines and eomesodermin (Eomes) *p<0.05; **p<0.01; two-tailed t test. (D) RNAseq gene expression data from HLA-DR+ and HLA-DR− tumors were analyzed for differences in the human orthologues as in (C). E) The TCGA breast cancer dataset were utilized to explore correlations between HLA-DRA mRNA and T cell chemo-kines across over 1000 patients. A correlation matrix was generated showing the Pearson's correlation coefficient across each gene pair. F) Cultures MMTV-neu tumor cells (cell line) expressing Ciita or vector control were analyzed by quantitative real-time PCR for tumor-specific changes in T cell-recruiting chemokines. *p<0.05; **p<0.01; ***p<0.001.

FIG. 9 shows that tumors escaping immunologic rejection in enforced-MHC-II expressing MMTV-neu tumors retain MHC-II expression. NanoString analysis of MHC-I and MHC-II gene expression in pMX-puro and pMX-Ciita transduced tumor cells from tumors harvested following tumor.

FIGS. 10A-B show higher eomesodermin expression in MHC-II+ tumors. A) NanoString analysis of eomesodermin gene expression in pMX-puro and pMX-Ciita transduced tumor cells from tumors harvested following tumor establishment. B) Eomesodermin gene expression as measured by RNAseq in melanoma and lung cancer patients, stratified by MHC-II status. **p<0.01; Mann Whitney U test.

FIG. 11 shows tumors escaping immunologic rejection in enforced MHC-II-expressing MMTV-neu tumors demonstrate higher CXCR3+ infiltrates. NanoString analysis of eomesodermin gene expression in pMX-puro and pMX-Ciita transduced tumor cells from tumors harvested following tumor establishment. **p<0.01 two-sample t test.

FIGS. 12A-E show combinatorial activity of PD-1 and Lag-3 neutralizing antibodies in MHC-II+ tumors. (A) Schematic of experimental strategy. On Day −10, 1e⁶ MMTV-neu cells transduced with pMX-puro or pMX-Ciita were implanted by orthotopic injection in the #4 mammary fat pad of wildtype FVB/n mice. Seven days later, a subset of mice were sacrificed for flow cytometry analysis. On day 0, therapy was initiated consisting of twice weekly IP injections of anti-IgG control, anti-PD-1, or anti-PD-1+anti-Lag-3, which continued for 2 weeks. Tumors were monitored for growth over the next 39 days. (B) Schematic for analyzing tumor and lymphoid compartments by flow cytometry. (C) Tumor growth curves for treated mice; CR: complete response; **p<0.05 by χ² test across treatment groups in Ciita-expressing tumors. (D) Flow cytometry analysis of PD-1+/Lag-3+ lymphocytes by total CD3+ compartment, or by CD3+/CD4+ or CD3+CD8+ compartments in lymphoid tissues at 7 days after injection. **p<0.01, two tailed Mann Whitney U test (E) Flow cytometry analysis of PD-1+/Lag-3+ lymphocytes in tumors (TILs). *p<0.05, Mann Whitney U test.

FIGS. 13A-J show that the alternative MHC-II receptor FCRL6 is an inhibitor of T cell and NK cell activity, is enriched in MHC-II+ tumors, and is associated with acquired resistance to anti-PD-1. (A) NK-92 and K562 transductants were stained with anti-FCRL6 or anti-HLA-DR, respectively. K562 vector (top), K562 DRa+b1 (middle), or K562 CIITA (bottom) transductants were cultured with NK-92 FCRL6 transductants (top lines), NK-92 vector (middle lines), or parental NK-92 cells (bottom lines) at various effector to target (E:T) ratios and assayed for K562 cytotoxicity in 51Cr release assays. Experiments were performed in triplicate; lines represent the mean±s.d.; n=4; P-values were calculated using Student's t-test. (B) Blood mononuclear cells were cultured for 6 d in the presence of the CEF peptide pool with anti-FCRL6, anti-PD-L1, or an isotype-matched control mAb. Cells were collected, re-stimulated with CEF for 6 h, and analyzed for intracellular cytokine production by flow cytometry. CD8+ T cells from one representative donor were gated, and the percentages of cells staining positive for the indicated cytokines are shown in the quadrants of each dot plot. (C) Paired data points from healthy donors (n=12 for IFNγ; n=9 for TNFα) are indicated by lines and statistical significance was determined using the Wilcoxon signed-rank test. (D) mRNA expression from RNAseq data for FCRL3 and FCRL6 in MHC-II+ and MHC-II-melanoma and lung cancers. (E) mRNA expression from RNAseq data for FCRL3 and FCRL6 in untreated and post-progression/relapse specimens. (F) Quantification of IHC for FCRL6+ TILs in pairs of melanomas before and after resistance to anti-PD-1 therapy. MHC-II+ tumors are colored in red. (G) MHC-II/HLA-DR AQUA scores in triple-negative breast cancers stratified by the presence or absence of FCRL6+ lymphocytes (H) Co-expression of Lag-3 and FCRL6 on TILs in post-neoadjuvant triple-negative breast cancers (I) MHC-II/HLA-DR AQUA scores in triple-negative breast cancers stratified by the presence or absence of FCRL6+ lymphocytes and Lag-3+ lymphocytes. (J) Fraction of CD8+ lymphocytes expressing granzyme-B (AQUA), stratified by presence or absence of FCRL6+ lymphocytes (IHC), Lag-3+ lymphocytes (IHC), or total tumor microenvironment PD-L1 expression (AQUA).

FIGS. 14A-C show a gating strategy for the studies described herein. A) Pulse geometry for identifying/enriching single-cell lymphocytes. B) Identification of CD4/CD8+ T cells. C) Fluorescence-minus-one controls for PD-1+CD3 cells and Lag3+CD3 cells (Gated on CD3+ cells for FMO controls). Left—CD3+ cells with no PD-1 or Lag3 antibody. Middle—CD3+ cells with only PD-1 antibody. Right—CD3+ cells with only Lag3 antibody.

FIG. 15 show the association of MHC-II receptor gene expression with degree of MHC-II positivity on tumor cells. RNAseq TPM counts for CD233/LAG-3 and FCRL6 were regressed and correlated to the fraction of HLA-DR+ tumor cells in the same specimen.

FIG. 16 shows representative FCRL6 IHC staining. Human spleen and lymph node tissue sections were used to validate IHC staining for FCRL6, according to the reported methods.

FIG. 17 shows a proposed mechanism for selective adaptive resistance to anti-PD-1-targeted immunotherapy in MHC-II+ tumors.

FIG. 18 shows that there was no association of FCRL6+ TILs and MHC-I (HLA-A) expression on tumor cells. No association was detected between the presence of FCRL6+ TILs and HLA-A expression on tumor cells (H-score; intensity [0-3+]* fraction of tumor cells staining positive).

### DETAILED DESCRIPTION

Molecular drivers of therapeutic resistance to cancer treatments are incompletely characterized. Described resistance mechanisms include downregulation of antigen machinery by somatic mutations in JAK/STAT pathways, alternative immune checkpoint expression, loss or lack of immunogenic neoantigens, and tumor intrinsic gene expression programs involving angiogenesis and wound healing. Identifying effective therapeutic strategies to overcome mechanisms of resistance and characterizing novel drivers remain critical unmet needs.

As shown herein, through dedicated immunohistological and RNA sequencing analyses of MHC-II phenotypes in tumors, patterns of inflammation present in those tumors expressing MHC-II were identified. MHC-II expression on tumor cells promotes anti-tumor immunity and facilitates the recruitment of CD4+ T cells, which corresponds to increased expression of CXCR3-binding T cell-recruiting cytokines. As tumors adapt to this microenvironment, either during cancer progression or during treatment with immunotherapies targeting the PD-1/L1 axis, they acquire immunosuppressive signals through alternative checkpoints that antagonize MHC-II expression, such as LAG-3.

Also shown is that another MHC-II receptor, Fc receptor-like 6 (FCRL6), is present in the tumor microenvironment of MHC-II+ tumors and is subsequently upregulated following progression on PD-1-directed immunotherapy. The studies provided herein show that, like Lag-3, FCRL6 provides an immunosuppressive signal that directly represses natural killer cell cytotoxic activity and effector T cell cytokine secretion when it engages MHC-II. Therefore, FCRL6 is a novel checkpoint and previously unrecognized immunotherapy target. Collectively, these data show a novel mechanism whereby an intrinsic tumor phenotype drives a unique pattern of adaptive resistance to immunotherapy, for example, resistance to anti-PD-1 or anti-PDL-1 therapy. FCRL6 is overexpressed in patients receiving anti PD-1 therapy indicating that anti-FCRL6 therapy can be used to treat cancer. For example, anti-FCRL6 therapy can be used to treat a cancer associated with increased expression of FCRL6. In particular, inhibition or blockade of FCRL6 can be used to overcome resistance to anti-PD-1 or anti-PDL-1 therapy.

Provided herein is a method of reducing resistance to an anti-programmed cell death PD-1 or an anti-PDL-1 therapy in a subject with cancer comprising administering to the subject with cancer an effective amount of an FCRL6 inhibitor, wherein the subject has been treated with an anti-PD1 therapy or an anti-PDL-1 therapy.

As used throughout, an anti-PD1 therapy or an anti-PDL-1 therapy is a therapy that inhibits the interaction between PD-1 and PDL-1 or the interaction between PD-1 or PDL-1 and other binding partners so as to remove T-cell dysfunction resulting from signaling on the PD-1/PD ligand signaling pathway. Anti-PD-1 or anti-PDL-1 therapy can be used to restore or enhance T-cell function (e.g., proliferation, cytokine production, target cell killing). In some examples, an anti-PDL-2 therapy can be used to inhibit or disrupt PD-1 signaling.

As used herein, an anti-PD1 therapy or an anti-PDL-1 therapy can be a chemical, small molecule, drug, protein, cDNA, antibody, a nucleic acid or any other compound. In some examples, the anti-PD-1 or anti-PDL-1 therapy is an immunotherapy comprising administration of an anti-PD-1 or an anti-PDL-1 antibody.

As used throughout, the term antibody encompasses, but is not limited to, whole immunoglobulin (i.e., an intact antibody) of any class, including polyclonal and monoclonal antibodies. Fragments of anti-FCRL6 antibodies that retain the ability to bind FCRL6 can also be used in any of the methods taught herein. Similarly, fragments of anti-PD-1 antibodies that retain the ability to bind to PD-1 and fragments of anti-PDL-1 antibodies that retain the ability to bind to PDL-1 can be used in any of the methods provided herein. Similarly, fragments of anti-LAG3 antibodies that retain the ability to bind to LAG3 can be used in any of the methods provided herein. Also useful in the methods provided herein are conjugates of antibody fragments and antigen binding proteins (single chain antibodies) as described, for example, in U.S. Pat. No. 4,704,692, the contents of which are hereby incorporated by reference in their entirety. Bispecific antibodies can also be used. Examples of anti-PD-1 antibodies include, but are not limited to, nivolumab (Opdivo®), pembrolizumab (Keytruda®), pidilizumab and BMS-936559. Examples of anti-PDL-1 antibodies include, but are not limited to, atezolizumab (Tecentriq®), durvalumab (Imfinzi®) and avelumab (Bavencio®).

As used herein, a nucleic acid that specifically inhibits or decreases expression and/or PD-1 or PDL-1 activity can be an antisense oligonucleotide, an siRNA, a morpholino, a locked nucleic ac, aid (LNA), an miRNA, a bridged nucleic acid (BNA), a peptide nucleic acid (PNA), a short hairpin RNA (shRNA), an ethylene-bridged nucleic acid (ENA), a 2′-O-methyl (2-OMe) modified RNA, a 2′-O-methoxyethyl (2-MOE) modified RNA, a hexitol nucleic acid, and/or an oligonucleotide with a phosphorothioated backbone.

As used throughout, cancer refers to any cellular disorder in which the cells proliferate more rapidly than normal tissue growth. A proliferative disorder includes, but is not limited to, neoplasms, which are also referred to as tumors. A neoplasm can include, but is not limited to, pancreatic cancer, breast cancer, brain cancer (e.g., glioblastoma), lung cancer, a central nervous system cancer, prostate cancer, colorectal cancer, head and neck cancer, ovarian cancer, thyroid cancer, renal cancer, bladder cancer, adrenal cancer and liver cancer A neoplasm can be a solid neoplasm (e.g., sarcoma or carcinoma) or a cancerous growth affecting the hematopoietic system. In some examples, the cancer is a triple negative (estrogen receptors negative (ER−), progesterone receptors negative (PR−) and HER2 negative (HER2−)) breast cancer. Examples of hematopoietic malignanacies include, but are not limited to, myelomas, leukemias, lymphomas (Hodgkin's and non-Hodgkin's forms), T-cell malignancies, B-cell malignancies, and lymphosarcomas.

As used herein, a PD1-mediated resistant cancer is a cancer that is resistant, i.e., less responsive as compared to a control, to treatment with an agent that disrupts the PD1/PD ligand signaling pathway. In any of the methods provided herein, the resistance can be primary resistance where a cancer does not respond to an anti-PD-1 or an anti-PDL-1 therapy, for example, an immunotherapy. The resistance can also be an adaptive immune resistance where a cancer is recognized by the immune system but it protects itself by adapting to the immune attack. Given the evolving nature of the immune/cancer cell interaction, this could clinically manifest as primary resistance, mixed responses or acquired resistance. In other examples, the resistance can be an acquired resistance to an anti-PD-1 or an anti-PDL-1 therapy in which a cancer initially responded to therapy but after a period of time, the cancer relapsed and progressed (See, for example, Sharma et al. “Primary, Adaptive, and Acquired Resistance to Cancer Immunotherapy,” Cell 168: 707-723 (2017), incorporated herein in its entirety by this reference.).

In the methods provided herein, a reduction in resistance refers to an increase in the responsiveness of the cancer to treatment. By increasing responsiveness, the efficacy of anticancer treatments, for example, anticancer treatments that the cancer was resistant to, can be increased. For example, a reduction in resistance can be an increase in responsiveness to an anti-PD-1 or anti-PDL-1 therapy. In some examples, a reduction in resistance can be an increase in responsiveness to an anti-PD-1/anti-LAG3 therapy. A reduction in resistance can be a 10%, 20%, 30%, 40%, 50%, 60%, 70%, 80%, 90%, 100% or any percent reduction in between these percentages, as compared to a subject that has been treated with an anti-PD-1 therapy or an anti-PDL-1 therapy and has not received treatment with an anti-FCRL6 inhibitor. A reduction in resistance can also be an increase in responsiveness to an anticancer therapy of about 10%, 20%, 30%, 40%, 50%, 60%, 70%, 80%, 90%, 100%, 200%, 300%, 400% or more, as compared to a subject that has been treated with an anti-PD-1 therapy or an anti-PDL-1 therapy and has not received treatment with an anti-FCRL6 inhibitor. In cases where a subject has an acquired resistance to therapy, a reduction in resistance can result in a decrease in the progression of a cancer that has relapsed. This decrease can be a 10%, 20%, 30%, 40%, 50%, 60%, 70%, 80%, 90%, 100% or any percent reduction in between these percentages, as compared to a subject that has been treated with an anti-PD-1 therapy or an anti-PDL-1 therapy and has not received treatment with an anti-FCRL6 inhibitor.

As used herein, an FCRL6 inhibitor is a molecule that decreases expression and/or activity of FCRL6. In any of the methods provided herein, the FCRL6 inhibitor can be a chemical, small molecule, drug, protein (for example, a soluble, recombinant protein), cDNA, antibody, a nucleic acid or any other compound.

As used herein, a nucleic acid that specifically inhibits or decreases expression and/or FCRL6 activity can be an antisense oligonucleotide, an siRNA, a morpholino, a locked nucleic ac, aid (LNA), an miRNA, a bridged nucleic acid (BNA), a peptide nucleic acid (PNA), a short hairpin RNA (shRNA), an ethylene-bridged nucleic acid (ENA), a 2′-O-methyl (2-OMe) modified RNA, a 2′-O-methoxyethyl (2-MOE) modified RNA, a hexitol nucleic acid, and/or an oligonucleotide with a phosphorothioated backbone.

The anti-FCRL6 antibody, 1D8, described in the Examples, can also be used in any of the methods described herein.

In any of the methods provided herein, treatment with the anti-PD1 therapy or the anti-PDL-1 therapy can occur prior to or concurrently with administration of the FCRL6 inhibitor. In some examples, the anti-PD1 therapy or the anti-PDL-1 therapy is administered in combination with a second therapeutic agent, for example, an anti-LAG3 therapy, a chemotherapeutic agent, a kinase inhibitor or an anti-CTLA4 therapy, to name a few. For examples of combination therapies comprising an anti-PD-1 or anti-PDL-1 therapy and a second therapeutic agent, see, for example, Sharma et al. As used herein, combination therapy is defined as administration of two or more agents at or near the same time. Optionally, one or more compositions comprising the two or more agents can be administered to the subject. Optionally, the two or more agents are in the same composition, for example, a solution for injection or intravenous delivery.

Examples of anti-LAG3 therapy include anti-LAG3 antibodies, for example, BMS-986016 and GSK2831781. In examples where the anti-PD1 or anti-PDL-1 therapy is administered prior to the administration of the FCRL6 inhibitor, the anti-PD1 or anti-PDL-1 therapy can be administered minutes or hours prior to administration of the FCRL6. In some cases, the anti-PD1-1 or anti-PDL-1 inhibitor is administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days prior to the administration of the FCRL6 inhibitor. In some examples, the subject is treated with the anti-PD-1 or anti-PDL-1 therapy for months or years before administration of the FCRL6 inhibitor. In some examples, an anti-PD1 therapy or an anti-PDL-1 therapy can be re-administered to the subject after administration of the FCRL6 inhibitor with or without a second therapeutic agent, for example, hours, days, or months after administration of the FCRL6 inhibitor.

Any of the methods provided herein can further comprise administering a chemotherapeutic agent, prior to, concurrently or after administration of the FCRL6 inhibitor to the subject. Examples of chemotherapeutic agents include, but are not limited to adriamycin, dactinomycin, bleomycin, vinblastine, acivicin, aclarubicin, acodazole hydrochloride, acronine, adozelesin, aldesleukin, altretamine, ambomycin, ametantrone acetate, aminoglutethimide, amsacrine, anastrozole, anthramycin, asparaginase, asperlin, azacitidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzelesin, cedefingol, chlorambucil, cirolemycin, cisplatin, cladribine, crisnatol mesylate, cyclophosphamide, cytarabine, dacarbazine, daunorubicin hydrochloride, decitabine, dexormaplatin, dezaguanine, dezaguanine mesylate, diaziquone, doxorubicin, doxorubicin hydrochloride, droloxifene, droloxifene citrate, dromostanolone propionate, duazomycin, edatrexate, eflornithine hydrochloride, elsamitrucin, enloplatin, enpromate, epipropidine, epirubicin hydrochloride, erbulozole, esorubicin hydrochloride, estramustine, estramustine phosphate sodium, etanidazole, etoposide, etoposide phosphate, etoprine, fadrozole hydrochloride, fazarabine, fenretinide, floxuridine, fludarabine phosphate, fluorouracil, flurocitabine, fosquidone, fostriecin sodium, gemcitabine, gemcitabine hydrochloride, hydroxyurea, idarubicin hydrochloride, ifosfamide, ilmofosine, interleukin II (including recombinant interleukin II, or rIL2), interferon alfa-2a, interferon alfa-2b, interferon alfa-nl, interferon alfa-n3, interferon beta-1a, interferon gamma-1 b, iproplatin, irinotecan hydrochloride, lanreotide acetate, letrozole, leuprolide acetate, liarozole hydrochloride, lometrexol sodium, lomustine, losoxantrone hydrochloride, masoprocol, maytansine, mechlorethamine hydrochloride, megestrol acetate, melengestrol acetate, melphalan, menogaril, mercaptopurine, methotrexate, methotrexate sodium, metoprine, meturedepa, mitindomide, mitocarcin, mitocromin, mitogillin, mitomalcin, mitomycin, mitosper, mitotane, mitoxantrone hydrochloride, mycophenolic acid, nocodazole, nogalamycin, ormaplatin, oxisuran, paclitaxel, pegaspargase, peliomycin, pentamustine, peplomycin sulfate, perfosfamide, pipobroman, piposulfan, piroxantrone hydrochloride, plicamycin, plomestane, porfimer sodium, porfiromycin, prednimustine, procarbazine hydrochloride, puromycin, puromycin hydrochloride, pyrazofurin, riboprine, rogletimide, safingol, safingol hydrochloride, semustine, simtrazene, sparfosate sodium, sparsomycin, spirogermanium hydrochloride, spiromustine, spiroplatin, streptonigrin, streptozocin, sulofenur, talisomycin, tecogalan sodium, tegafur, teloxantrone hydrochloride, temoporfin, teniposide, teroxirone, testolactone, thiamiprine, thioguanine, thiotepa, tiazofurin, tirapazamine, toremifene citrate, trestolone acetate, triciribine phosphate, trimetrexate, trimetrexate glucuronate, triptorelin, tubulozole hydrochloride, uracil mustard, uredepa, vapreotide, verteporfin, vinblastine sulfate, vincristine sulfate, vindesine, vindesine sulfate, vinepidine sulfate, vinglycinate sulfate, vinleurosine sulfate, vinorelbine tartrate, vinrosidine sulfate, vinzolidine sulfate, vorozole, zeniplatin, zinostatin, zorubicin hydrochloride.

Any of the methods provided herein can optionally further include administering radiation therapy to the subject. Any of the methods provided herein can optionally further include surgery.

Also provided herein is a method of treating or preventing a PD-1 mediated resistant cancer in a subject that has been treated with an anti-PD1 therapy or an anti-PDL-1 therapy comprising administering to the subject an effective amount of a FCRL6 inhibitor.

As used throughout, by subject is meant an individual. Preferably, the subject is a mammal such as a primate, and, more preferably, a human. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical formulations are contemplated herein.

Throughout, treat, treating, and treatment refer to a method of reducing or delaying one or more effects or symptoms of cancer. In some examples, the cancer is a PD-1 mediated resistant cancer. Treatment can also refer to a method of reducing the underlying pathology rather than just the symptoms. The effect of the administration to the subject can have the effect of, but is not limited to, reducing one or more symptoms (e.g., reduced pain, reduced size of the tumor, etc.) of the cancer, an increase in survival time, a decrease or delay in metastasis, enhancing T cell function (e.g., proliferation, cytokine production, tumor cell killing), a reduction in the severity of the cancer (e.g., reduced rate of growth of a tumor or rate of metastasis), increasing latency between symptomatic episodes, decreasing the number or frequency of relapse episodes, the complete ablation of the cancer or a delay in the onset or worsening of one or more symptoms. For example, a disclosed method is considered to be a treatment if there is about a 10% reduction in one or more symptoms of the disease in a subject when compared to the subject prior to treatment or when compared to a control subject or control value. Thus, the reduction can be about a 10, 20, 30, 40, 50, 60, 70, 80, 90, 100%, or any amount of reduction in between.

As used throughout by prevent, preventing, or prevention is meant a method of precluding, delaying, averting, obviating, forestalling, stopping, or hindering the onset, incidence, severity, or recurrence of a PD1-mediated resistant cancer. For example, the disclosed method is considered to be a prevention if there is a reduction or delay in onset, incidence, severity, or recurrence of a PD1-mediated resistant cancer or one or more symptoms of a PD1-mediated resistant cancer (e.g., relapse, disease progression, increase in tumor size, metastasis) in a subject treated with an anti-PD-1 or an anti-PDL-1 therapy as compared to control subjects treated with an anti-PD-1 or an anti-PDL-1 therapy that did not receive an anti-FCRL6 inhibitor. The disclosed method is also considered to be a prevention if there is a reduction or delay in onset, incidence, severity, or recurrence of PD1-mediated resistant cancer or one or more symptoms of a PD-1-mediated resistant cancer in a subject after receiving an FCRL6 inhibitor as compared to the subject's progression prior to receiving treatment. Thus, the reduction or delay in onset, incidence, severity, or recurrence of a PD1-mediated resistant cancer can be about a 10, 20, 30, 40, 50, 60, 70, 80, 90, 100%, or any amount of reduction in between.

Provided herein are methods of diagnosing a PD-1 mediated resistant cancer comprising detection of the levels of one or more biomarkers indicative of an acquired resistance to PD-1 therapy preferentially occurring in MHC-II+ tumors, for example, increased levels of FCRL6 and/or LAG3. Provided herein is a method of diagnosing and treating a PD-1-mediated resistant cancer in a subject that has been treated with an anti-PD1 therapy or an anti-PDL-1 therapy comprising the steps of a) obtaining a biological sample from a subject that has been treated with an anti-PD1 therapy or an anti-PDL-1 therapy; b) detecting the level of FCRL6 in the sample; c) diagnosing the subject with a PD-1 mediated resistant cancer when the level of FCRL6 in the sample is increased as compared to a control sample; and d) administering an effective amount of an FCRL6 inhibitor.

Also provided is a method of diagnosing and treating a PD-1-mediated resistant cancer in a subject that has been treated with an anti-PD1 therapy or an anti-PDL-1 therapy comprising the steps of a) obtaining a biological sample from a subject that has been treated with an anti-PD1 therapy or an anti-PDL-1 therapy; b) detecting the level of FCRL6 and/or the level of LAG3 in the sample; c) diagnosing the subject with a PD-1 mediated resistant cancer when the level of FCRL6 and/or the level of LAG3 in the sample is increased as compared to a control sample; and d) administering an effective amount of an anti-LAG3 therapy and/or an FCRL6 inhibitor to the subject.

Also provided is a method of diagnosing and treating a PD-1-mediated resistant cancer in a subject that has been treated with an anti-PD1 therapy or an anti-PDL-1 therapy comprising: a) obtaining a biological sample from a subject that has been treated with an anti-PD1 therapy or an anti-PDL-1 therapy; b) detecting the level of FCRL6 and the level of LAG3 in the sample; c) diagnosing the subject with a PD-1 mediated resistant cancer when the level of FCRL6 and the level of LAG3 in the sample are increased as compared to a control sample; d) administering an effective amount of an anti-LAG3 therapy and/or an FCRL6 inhibitor to the subject.

Any of the methods of diagnosing and treating a PD-1-mediated resistant cancer can further comprise detecting MHC-II expression levels or detecting MHC-II+ T cells in the sample. In some examples, an increased level of MHC-II+ and/or FCRL6 is indicative of responsiveness to FCRL6 inhibition. In the methods of diagnosing and treating, the level of FCRL6 and/or LAG3 in the sample can be compared to the level of FCRL6 and/or LAG3 in a control sample from a subject treated with an anti-PD1 or anti-PDL-1 therapy that did not develop resistance to the anti-PD1 or anti-PDL-1 therapy. The sample can be from the same subject at a time when the subject was responsive to the anti-PD1 or anti-PDL-1 therapy and had not developed resistance or from a different subject. Alternatively, depending on the type of cancer, the level of FCRL6 and/or LAG3 can be compared to a control value for the level of FCRL6 and/or LAG3 associated with the cancer when resistance to anti-PD1 or anti-PDL-1 therapy does not develop.

Any of the methods of treating a PD-1-mediated resistant cancer can further comprise administering an anti-PD-1 or an anti-PDL-1 therapy, with or without a second therapeutic agent to the subject. The anti-PD-1 or anti-PDL-1 therapy can be administered concurrently with the FCRL6 inhibitor and/or the anti-LAG3 inhibitor or after administration of the FCRL6 inhibitor and/or the anti-LAG3 inhibitor. In some examples, an anti-PD1/anti FCRL6 combination therapy is administered to the subject. In other examples, an anti-PD-1/anti-LAG3 combination therapy is administered to the subject. In some examples, an anti-FCRL6/anti-LAG3 combination therapy is administered to the subject.

Any of the methods of treating a PD-1 mediated resistant cancer can further comprise administering an anti-CTLA4 antibody, an anti-PDL-1 antibody, an anti-PDL2 antibody, a kinase inhibitor or a chemotherapeutic agent, to name a few. Therefore, the methods of treating a PD-1 mediated resistant cancer can comprise administering an FCRL6 inhibitor and one or more of an anti-PD1 antibody, an anti-LAG3 antibody, anti-CTLA4 antibody, an anti-PDL-1 antibody, an anti-PDL2 antibody, a kinase inhibitor or a chemotherapeutic agent.

Abiological sample can be any sample obtained from an organism. Examples of biological samples include body fluids and tissue specimens. For example, the sample can be a tissue biopsy, for example, a tumor biopsy. The source of the sample may also be physiological media such as blood, serum, plasma, cerebral spinal fluid, breast milk, pus, tissue scrapings, washings, urine, feces, tissue, such as lymph nodes, spleen or the like. The term tissue refers to any tissue of the body, including blood, connective tissue, epithelium, contractile tissue, neural tissue, and the like.

In the methods provided herein, the level of FCRL6 and/or LAG3 in the sample can be determined by measuring the amount of a mRNA encoding FCRL6 and/or LAG3 in a sample using methods standard in the art for quantitating nucleic acids. These include, but are not limited to, in situ hybridization, quantitative PCR, RT-PCR, Taqman assay, Northern blotting, ELISPOT, dot blotting, etc., as well as any other method now known or later developed for quantitating the amount of a nucleic acid in a cell or released from a cell. The amount of FCRL6 or LAG3 protein or a fragment thereof in a sample, can be determined by methods standard in the art for quantitating proteins such as densitometry, absorbance assays, fluorometric assays, Western blotting, ELISA, radioimmunoassay, ELISPOT, immunoprecipitation, immunofluorescence (e.g., FACS), immunohistochemistry, etc., as well as any other method now known or later developed for quantitating a specific protein in or produced by cells in a sample. In some examples, the level of FCRL6 can be detected by detecting the level of FCRL6+T or NK cells in circulation using flow cytometry. Imaging techniques can be used to detect FCRL6 or LAG3. For example imaging techniques that use target-specific contrast agents can be used to detect FCRL6 protein or LAG3 protein in a sample or in a subject. In another example, an imaging agent, such as a labeled binding protein, antibody or a functional fragment thereof that specifically binds to FCRL6 or LAG3 can be administered to a subject. The imaging agent is administered in an amount effective for diagnostic use in a mammal such as a human. The localization and accumulation of the imaging agent is then detected after it has bound to FCRL6 or LAG3 present in the sample or the subject. The localization and accumulation of the imaging agent can be detected by radionucleide imaging, radioscintigraphy, nuclear magnetic resonance imaging, computed tomography, positron emission tomography, computerized axial tomography, X-ray or magnetic resonance imaging method, fluorescence detection and/or chemiluminescent detection. The imaging agent can be labeled with a radionucleide or a non-radioactive indicator.

The term effective amount, as used throughout, is defined as any amount of an agent (for example, an anti-PD-1 agent, anti-PDL-1 agent, anti-LAG3 agent, anti-FCRL6 agent, a chemotherapeutic agent, etc.) necessary to produce a desired physiologic response. Exemplary dosage amounts for a mammal include doses from about 0.5 to about 200 mg/kg of body weight of active compound per day, which may be administered in a single dose or in the form of individual divided doses, such as from 1 to 4 times per day can be used. Alternatively, the dosage amount can be from about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 0.5 to about 15 mg/kg of body weight of active compound per day, about 0.5 to about 10 mg/kg of body weight of active compound per day, about 0.5 to about 5 mg/kg of body weight of active compound per day, about 1 to about 20 mg/kg of body weight of active compound per day, about 1 to about 10 mg/kg of body weight of active compound per day, about 1 to about 5 mg/kg of body weight of active compound per day, about 20 mg/kg of body weight of active compound per day, about 10 mg/kg of body weight of active compound per day, or about 5 mg/kg of body weight of active compound per day. One of skill in the art would adjust the dosage as described below based on specific characteristics of the agent and the subject receiving it.

Effective amounts and schedules for administering the agent can be determined empirically and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, unwanted cell death, and the like. Generally, the dosage will vary with the type of inhibitor, the species, age, body weight, general health, sex and diet of the subject, the mode and time of administration, rate of excretion, drug combination, and severity of the particular condition and can be determined by one of skill in the art. The dosage can be adjusted by the individual physician in the event of any contraindications. Dosages can vary, and can be administered in one or more dose administrations daily, for one or several days. Guidance can be found in the literature for appropriate dosages for given classes of pharmaceutical products.

Any of the agents described herein can be provided in a pharmaceutical composition. These include, for example, a pharmaceutical composition comprising a therapeutically effective amount of one or more agents and a pharmaceutical carrier.

Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the agent described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected agent without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material known in the art for use in pharmaceutical formulations. The choice of a carrier for use in a composition will depend upon the intended route of administration for the composition. The preparation of pharmaceutically acceptable carriers and formulations containing these materials is described in, e.g., Remington: The Science and Practice of Pharmacy, 22nd edition, Loyd V. Allen et al, editors, Pharmaceutical Press (2012).

Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICSm (BASF; Florham Park, N.J.).

Compositions containing one or more of the agent(s) described herein suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof are admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, such as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

The compositions are administered in any of a number of ways depending on whether local or systemic treatment is desired and on the area to be treated. Any of the compositions described herein can be delivered by any of a variety of routes including by injection (e.g., subcutaneous, intramuscular, intravenous, intra-arterial, intraperitoneal), by continuous intravenous infusion, cutaneously, dermally, transdermally, orally (e.g., tablet, pill, liquid medicine, edible film strip), by implanted osmotic pumps, by suppository, or by aerosol spray. Routes of administration include, but are not limited to, topical, intradermal, intrathecal, intralesional, intratumoral, intrabladder, intravaginal, intra-ocular, intrarectal, intrapulmonary, intracranial, intraventricular, intraspinal, dermal, subdermal, intra-articular, placement within cavities of the body, nasal inhalation, pulmonary inhalation, impression into skin, and electroporation.

In an example in which a nucleic acid is employed the nucleic acid can be delivered intracellularly (for example by expression from a nucleic acid vector or by receptor-mediated mechanisms), or by an appropriate nucleic acid expression vector which is administered so that it becomes intracellular, for example by use of a retroviral vector (see U.S. Pat. No. 4,980,286), or by direct injection, or by use of microparticle bombardment (such as a gene gun; Biolistic, Dupont), or coating with lipids or cell-surface receptors or transfecting agents, or by administering it in linkage to a homeobox-like peptide which is known to enter the nucleus (for example Joliot et al., Proc. Nat. Acad. Sci. USA 1991, 88:1864-8). Nucleic acid carriers also include, polyethylene glycol (PEG), PEG-liposomes, branched carriers composed of histidine and lysine (HK polymers), chitosan-thiamine pyrophosphate carriers, surfactants, nanochitosan carriers, and D5W solution. The present disclosure includes all forms of nucleic acid delivery, including naked DNA, plasmid and viral delivery, integrated into the genome or not. Physical transduction techniques can also be used, such as liposome delivery and receptor-mediated and other endocytosis mechanisms (see, for example, Schwartzenberger et al., Blood 87:472-478, 1996) to name a few examples. This invention can be used in conjunction with any of these or other commonly used gene transfer methods.

Effective doses for any of the administration methods described herein can be extrapolated from dose-response curves derived from in vitro or animal model test systems.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

A subset of breast cancers express major histocompatibility complex II (MHC-II), correlating with enhanced immune infiltration. In other tumor types, MHC-II expression on tumor cells predicts clinical response to checkpoint inhibition. Whether MHC-II expression in breast tumors directly enhances anti-tumor immunity, or whether it is an epiphenomenon was determined.

To determine the functional effects of MHC-II on tumor cells, isogenic murine breast tumor cells with enforced MHC-II expression were generated and their ability to generate tumors in syngeneic mice, the impact on immunity, and their response to checkpoint inhibition was determined as set forth below.

### Methods

### Patient Samples

For melanoma/lung cohorts, 58 patient samples and data were procured based on availability of tissue and were not collected according to a pre-specified power analysis. These samples included 48 melanoma samples and 10 lung cancer samples. Included in these 58 samples were 8 relapse specimens (50 baseline pre-anti-PD-1 therapy), of which 5 were matched to a pre-anti-PD-1 therapy sample. Of these, 3 pairs were successfully RNA sequenced, 5 pairs were successfully scored for LAG-3 IHC and 4 had sufficient tissue for FCRL6 IHC.

All patients provided informed written consent on IRB approved protocols (Vanderbilt IRB #030220 and 100178). Tumor samples for the TMA and for the HLA-DR staining cohort were obtained from tumor biopsies or tumor resections obtained for clinical purposes. Samples were obtained within 2 years of start of anti-PD-1/PD-L1 therapy (nivolumab, pembrolizumab, atezolizumab). Only patients with available tumor samples and evaluable responses were included. In cases where multiple tissues were available for the same patient, the evaluable sample collected closest to PD-1 therapy was utilized for scoring. Clinical characteristics and objective response data were obtained by retrospective review of the electronic medical record. All responses were investigator assessed, RECIST defined responses or (in a single case) prolonged stable disease with clinical benefit lasting >3 years.

For the triple negative breast cancer (TNBC) cohort, one-hundred-eleven (112) surgically-resected tumor samples were from patients with TNBC diagnosed and treated with neoadjuvant chemotherapy at the Instituto Nacional de Enfermedades Neoplisicas in Lima, Pern. Clinical and pathological data were retrieved from medical records under an institutionally approved protocol (INEN 10-018). Tumors were determined as triple negative if they were negative for estrogen receptor, progesterone receptor and HER2 overexpression measured by IHC. The analysis of these samples has been previously described (Loi et al., “RAS/MAPK Activation Is Associated with Reduced Tumor-Infiltrating Lymphocytes in Triple-Negative Breast Cancer: Therapeutic Cooperation Between MEK and PD-1/PD-L1 Immune Checkpoint Inhibitors,” Clin Cancer Res 22: 1499-1509 (2016)).

### Immunohistochemistry and Immunofluorescence

For HLA-DR (Santa Cruz (Dallas, Tex.) [sc-53319]; 1:1000) and SOX10 (LsBio (Seattle, Wash.) [LS-C312170]; 1:30) dual IHC, tumor sections were stained overnight at 4° C. with both antibodies. Antigen retrieval was performed using citrate buffer (pH 6) using a Biocare Decloaking Chamber. The visualization system utilized was MACH2 (Biocare, Pacheco, Calif.) using DAB (Dako, Atlanta, Ga.) and Warp Red (Biocare), and counterstained with hematoxylin. HLA-DR scoring in the tumor compartment was performed as described in Johnson et al. (Nat. Commun. 7: 10582 (2016)). For FCRL6 (Clone 7B7³⁴; 1:100) and LAG-3 (Cell Signaling (Danvers, Mass.) [15372]; 1:200) detection, a citrate Buffer (pH 6) was used for antigen retrieval, with Envision for the visualization system. For CD4 and CD8 staining, slides were placed on a Leica Bond Max IHC stainer (Leica Biosystems, Wetzlar, Germany). All steps besides dehydration, clearing and coverslipping are performed on the Bond Max. Heat induced antigen retrieval was performed on the Bond Max using their Epitope Retrieval 2 solution for 20 minutes. Slides were incubated with anti-CD4 (PA0427, Leica, Buffalo Grove, Ill.) or anti-CD8 (MS-457-R7, ThermoScientific, Kalamazoo, Mich.) for one hour. The Bond Polymer Refine detection system was used for visualization. CD4 and CD8 were scored as % infiltrating CD4(+) or CD8(+) cells in the tumor area. HLA-DR immunofluorescence/AQUA and PD-L1 immunofluorescence/AQUA were performed as previously described (Loi et al.). Additional multiplexed immunohistochemistry was performed with sequential staining for CD4 (M7310 1:50, DAKO), CD8 (M7103 1:400, DAKO), Granzyme B (GrB-7 1:300, DAKO), amplified with EnVision+HRP Mouse (DAKO), detected with TSA-Cy5 (1:50, Perkin Elmer (Duluth, Ga.), Opal 540 (1:200, Perkin Elmer), and Opal 620 (1:200, Perkin Elmer) respectively. Additionally, staining included pan-cytokeratin (Z0622 1:200, DAKO) detected with goat-anti-rabbit Alexa488 (1:200, Life Technologies (Carlsbard, Calif.), and 4′,6-diamidino-2-phenylindole (DAPI). Digital imaging was performed on the Vectra 2 (Perkin Elmer) and expression of all cells CD4+, CD8+ or the percentage of CD4+ or CD8+ cells also expressing Granzyme B were quantified using AQUA® Technology (Navigate BioPharma Services, Inc., Carlsbad, Calif.).

### TCGA Data Analysis

TCGA data were accessed through the cBio data portal (Cerami et al. Cancer Discov. 2: 401-404 (2012)), or through the TCGA data portal for processed RNA-SEQ data analysis (TCGA. Comprehensive molecular portraits of human breast tumours. Nature 490, 61-70 (2012)).

### Cell Culture

MMTV-neu cells were isolated from primary mammary tumor cells growing in transgenic FVB/Nmice and passaged serially for >10 passages in DMEM/F12 media supplemented with 10% FBS, 20 ng/mL EGF, 500 ng/mL hydrocortisone, and 10 ng/mL insulin to generate an established cell line. Presence of rat neu (Western blot) in the cells is diagnostic for the authenticity of the cells and is performed on a regular basis. K562 cells were obtained from ATCC and maintained in Iscove's DMEM supplemented with 10% FCS, 100 units/mL penicillin and streptomycin, and 50 μM β-mercaptoethanol. NK-92 cells were obtained from ATCC and maintained according to their recommendations.

### Lentiviral/Retroviral Transduction

The murine Ciita open reading frame was obtained from Genecopoeia (Rockville, Md.), amplified by PCR with restriction sites and cloned into the multiple cloning site of pMX-puro. Retroviral production was performed in Amphopak cells. MMTV-neu cells were transduced as previously described (Balko et al. “Molecular profiling of the residual disease of triple-negative breast cancers after neoadjuvant chemotherapy identifies actionable therapeutic targets,” Cancer Discov 4, 232-245 (2014).

For NK-92 cell line transduction, an FCRL6 lentiviral vector (pEF.FCRL6) was generated by replacing the GFP gene in pEF.GFP (Zufferey et al. Self-inactivating lentivirus vector for safe and efficient in vivo gene delivery. J Virol 72: 9873-9880 (1998)), with full-length FCRL6 cDNA. pEF.FCRL6 and pEF.GFP lentiviruses were packaged in 293T cells for transduction, and stable transductants were FACS sorted for FCRL6 or GFP enrichment. HLA-DRα (DRA*0101), HLA-DR/31 (DRB1*040101), and CITA were subcloned into the pMX-PIE retroviral vector, which contains the GFP gene downstream of an internal ribosomal entry site (IRES), and used to transduce K562 cells as described previously (Ehrhardt et al. “The inhibitory potential of Fc receptor homolog 4 on memory B cells. Proc Natl Acad Sci USA 100, 13489-13494 (2003)). Prior to retroviral transduction, K562 cells were transiently transfected with the murine ecotropic receptor MCAT1 (a gift of Dr. Gotz Ehrhardt, University of Toronto) by electroporation. Doubly-transduced HLA-DRα+β1 cells were sorted for GFP expression and surface staining with the PE-labeled HK14 anti-HLA-DR mAb (Sigma, St. Louis, Mo.); singly transduced HLA-DRα, HLA-DRβ1, and CIITA cells were FACS sorted for GFP.

### Mouse Studies

MMTV-neu cells (1×10⁶ cells) were injected in the #4 mammary fat pad of syngeneic FVB/N mice. Following tumor establishment, when palpable tumors reached 100 mm³, mice were observed for tumor growth rate, spontaneous rejection, or in later studies, randomized to treatment groups or vehicle control. Murine α-PD-1 and α-Lag-3 blocking antibodies were purchased from BioXcell and injected intraperitoneally twice weekly at 100 μg/100 μL for a total of 2 weeks. Control IgG was also administered IP in vehicle-treated mice. Mice were monitored twice weekly for tumor formation and tumor burden (digital calipers) using the using the formula (length/2×width²).

### NanoStrin Analysis

NanoString analysis was performed on mouse tumors using the Pan-Cancer Immunology panel as previously described (Loi et al.). Briefly, single cross sections of FFPE tumors harvested at the human endpoint (2 cm² total tumor burden) were used for RNA preparation using the Maxwell-16 FFPE RNA kit (Promega, Madison Wis.) and 50 ng of total RNA>300 nt was used for input into nCounter hybridizations. Quality-control measures and normalization of data were performed using the nSolver analysis package, and R.

### Flow Cytometry

Flow cytometry was performed on an Attune NxT Flow Cytometer (Thermo-Fisher, Waltham, Mass.). For basic immunophenotyping, freshly dissociated tumors were stained with viability-dye (Zombie Aqua; Biolegend), and fluorophore-conjugated anti-CD3 (17A2; A488), anti-CD4 (GK1.5; APC), anti-CD8 (53-6.7; PE-Cy7), anti-PD-1 (29F.1A12; BV711), and anti-Lag-3 (C9B7W; PE) (all from Biolegend). Additional antibodies utilized were anti-IA/IE (MHC-II; M5/114.15.2; A488) and anti-CD45 (30-F11; A700), all from Biolegend). All antibodies were titrated to optimal concentrations prior to staining.

### RNA Sequencing

Total RNA quality was assessed using the 2200 Tapestation (Agilent, Alpharetta, Ga.). At least 20 ng of DNase-treated total RNA having at least 30% of the RNA fragments with a size >200 nt (DV200) was used to generate RNA Access libraries (Illumina, San Diego, Calif.) following manufacturer's recommendations. Library quality was assessed using the 2100 Bioanalyzer (Agilent) and libraries were quantitated using KAPA Library Quantification Kits (KAPA Biosystems, Basel, Switzerland). Pooled libraries were subjected to 75 bp paired-end sequencing according to the manufacturer's protocol (Illumina HiSeq3000). Bcl2fastq2 Conversion Software (Illumina) was used to generate de-multiplexed Fastq files.

QC for the paired-end raw sequencing reads of all samples were performed using FastQC for the analysis of sequence quality, GC content, the presence of adaptors, overrepresented k-mers and duplicated reads. Sequencing reads were mapped to human reference genome GRCH38 (Release-85, Ensembl) using STAR 2.2.1 with 2-pass mapping. QC for read alignment and mapping was evaluated with RSeQC for sequencing saturation, mapped reads clipping profile, mapped read distribution, and coverage uniformity. The TPM (Transcripts per million) values were calculated using RSEM⁴⁵ and used to assess the global quality and reproducibility of the RNA-seq dataset and exported for downstream data analyses.

### Chromium-51 Release Assays

⁵¹Cr release experiments were performed as described previously (Whiteside et al. Measurement of cytotoxic activity of NK/LAK cells. Curr Protoc Immunol Chapter 7, Unit 7 18 (2001)). Briefly, 1×10⁶ K562 transductants were labeled for 1 h with 100 μCi/mL ⁵¹Cr (Perkin Elmer), washed, and plated at 1×10⁴ cells/well in 96 well round bottom plates. NK-92 cells were washed, resuspended in warm RPMI 1640 media containing 10% FCS, and plated with K562 cells at various effector to target ratios. Plates were spun at 1000 RPM for 1 min then cultured for 4 h at 37° C. Supernatants were collected, transferred to 96 well scintillant-coated plates (Perkin Elmer), and ⁵¹Cr activity was measured using a TopCount NXT instrument (Packard, Downers Grove, Ill.). Spontaneous release and total release were assessed from K562 cells cultured in media alone or 2% Triton X-100, respectively. Specific lysis was calculated as described previously (Whiteside et al.). To equalize culture conditions, each NK-92 cell line was seeded in fresh media at a concentration of 2×10⁵ cells/mL and cultured for 2 d prior to performing cytotoxicity experiments.

### Healthy Donor T Cell Assays

Freshly isolated PBMCs were cultured in RPMI 1640 media supplemented with 10% FCS, 100 units/mL penicillin and streptomycin, and 50 μM β-mercaptoethanol and a CEF peptide pool (Anaspec, Fremont, Calif.) at 0.4 μg/mL. Control IgG1 (Southern Biotechnology Associates, Birmingham, Ala.), anti-PD-L1 (eBioscience, Waltham, Mass.), and anti-FCRL6 (1D8) mAbs were added at 5 μg/mL to the initial cultures and on day 6 cells were collected, washed, and re-stimulated for 6 h with CEF at 2 μg/mL in the presence of brefeldin-A and monensin (Biolegend). Cells were then stained for surface markers, fixed and permeablized, cytoplasmically stained with anti-IFNγ APC and anti-TNFα PE (Biolegend), and analyzed with a Cyan flow cytometer.

### Statistical Analysis

Statistics were performed as indicated using R or GraphPad Prism (GraphPad Software, San Diego, Calif.). A p<0.05 was considered statistically significant for all studies. For paired analysis, a two-sample paired T test was utilized. Log-transformed data were utilized prior to analysis, where distributions demonstrated heteroscedasticity in linear form. For data with unequal variances, even after transformation, non-parametric equivalents were utilized. For multi-group comparisons, ANOVA was utilized with Tukey's post-hoc analysis to identify group-specific differences.

### Results

MHC-II-Positive (+) Tumors are Enriched with Gene Expression Patterns of Adaptive Immunity.

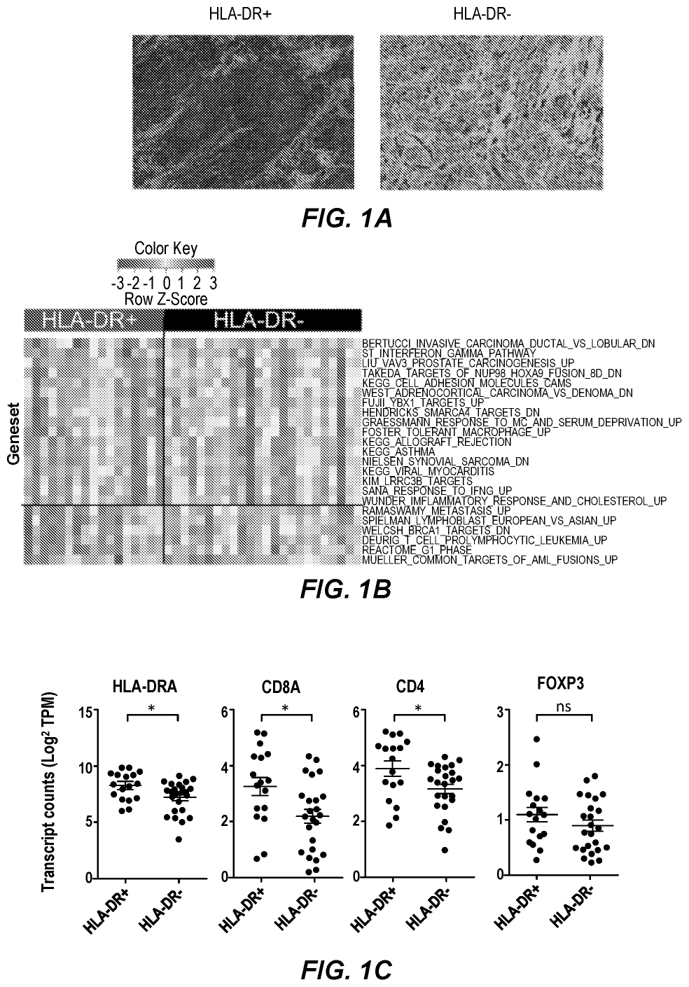

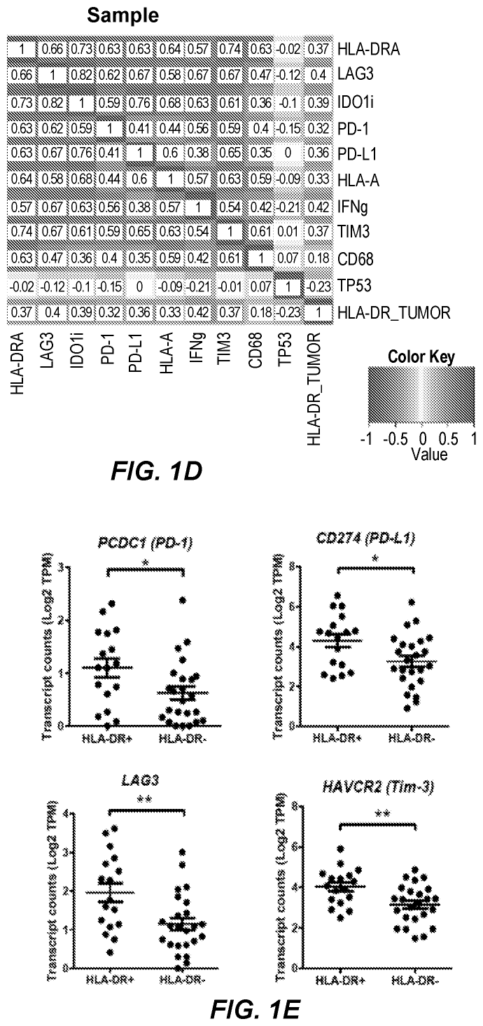

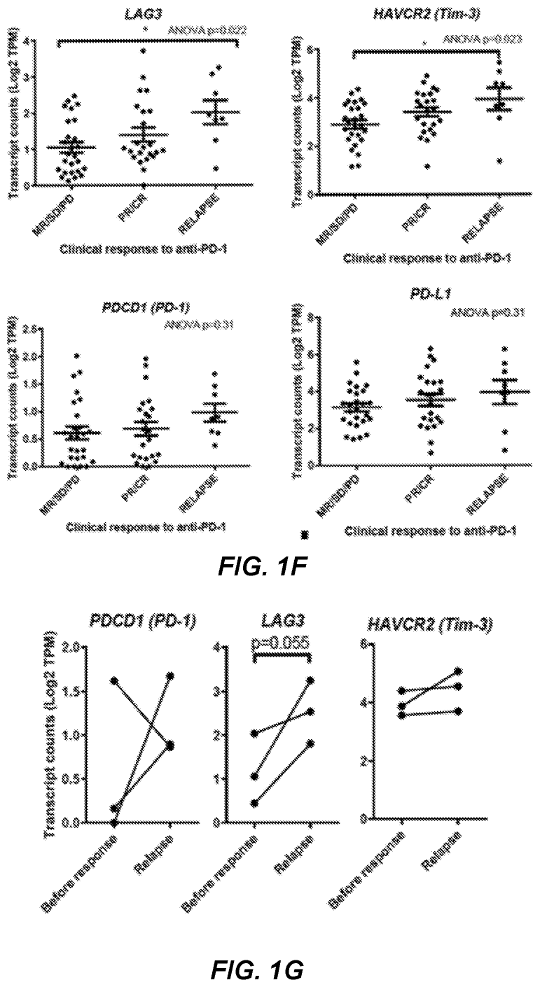

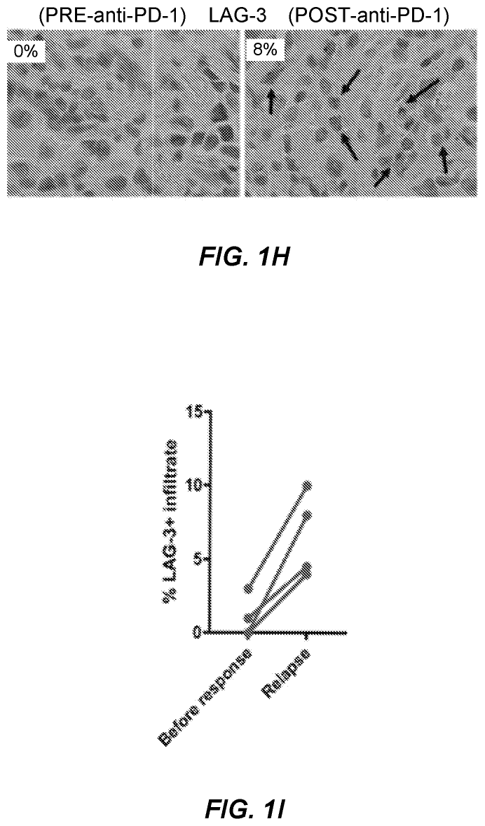

Melanomas with constitutive tumor cell autonomous MHC-II/HLA-DR expression are associated with high CD4 and CD8 infiltration and enhanced responses to PD-1-targeted immunotherapy (Johnson et al. “Melanoma-specific MHC-II expression represents a tumour-autonomous phenotype and predicts response to anti-PD-1/PD-L1 therapy. Nat Commun 7: 10582 (2016)). Furthermore, MHC-II+ melanoma cell lines (grown in the absence of stroma or IFN-γ-expressing cells) demonstrate intrinsic gene expression patterns of inflammation and autoimmunity (Johnson et al.). RNA-sequencing analysis was performed on a series of anti-PD-1-treated melanoma and non-small cell lung cancers (NSCLC) (n=58; including 50 pre-anti-PD-1 samples and 8 acquired resistance, post-anti-PD-1 samples) and scored tumor-specific HLA-DR expression by immunohistochemistry (IHC) (HLA-DR staining available on 41/58; FIG. 1A) prior to their treatment with PD-1-targeted immunotherapy. Tumors with at least 5% of tumor cells expressing cell-surface HLA-DR demonstrated similar gene set enrichment as observed in Johnson et al. The gene sets enriched (FDR<5%) in HLA-DR+ tumors included those associated with allograft rejection, viral myocarditis, auto-inflammatory disease (asthma) and IFN-γ response pathways (FIG. 1B). HLA-DR+ tumors had greater mRNA expression of MHC-II genes such as HLA-DRA, although the association was weak, reflecting the independent contribution of HLA-DR+ stroma to this measurement. HLA-DR+ tumors had higher CD8A and CD4 expression, without enhanced regulatory T cell markers, such as Foxp3 (FIG. 1C).

MHC-II+ Tumors are Associated with Higher Expression of Immune Checkpoints.

To explore the effects of tumor cell autonomous MHC-II expression on antigen presentation machinery and immune checkpoints, HLA-DR expression (scored by IHC) was correlated with genes associated with MHC-II (HLA-DRA), MHC-I (HLA-A), T cell repression (PD-1/PDCD1, PD-L1/CD274, IDO1, TIM-3/HAVCR2, and LAG3), T cell activation (IFNG), monocyte infiltration (CD68), and a ubiquitous marker (TP53) as a control (FIG. 1D). The expression of most immune-related genes correlated positively with one another. HLA-DR IHC expression, as scored only in the tumor compartment, also correlated with most immune genes, but less strongly with the myeloid marker CD68. Interestingly, all examined immune checkpoint genes also highly correlated with HLA-DR positivity, and the most significant of these was LAG3. These associations were also evident when stratifying tumors by MHC-II+>5% (FIG. 1E). Of particular interest in this analysis was the association of HLA-DR tumor cell positivity with LAG3, which competes with CD4 as a ligand for MHC-II, thereby suppressing MHC-II-mediated antigen presentation.

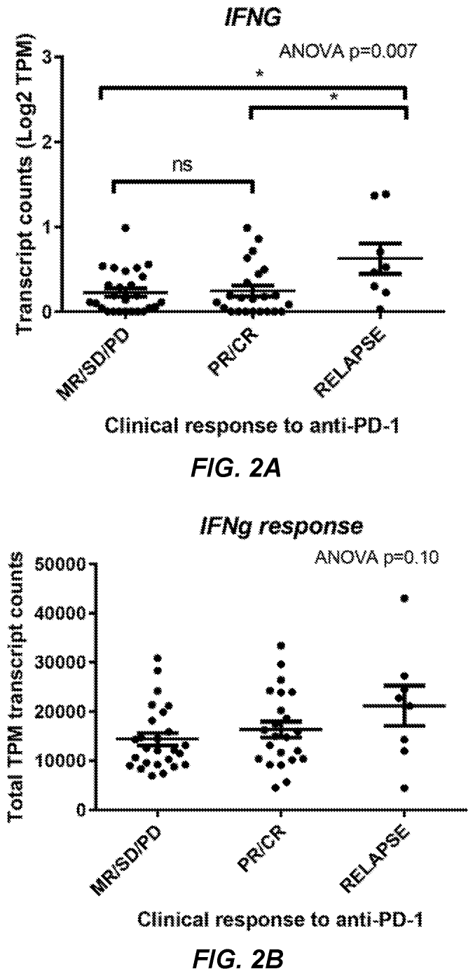

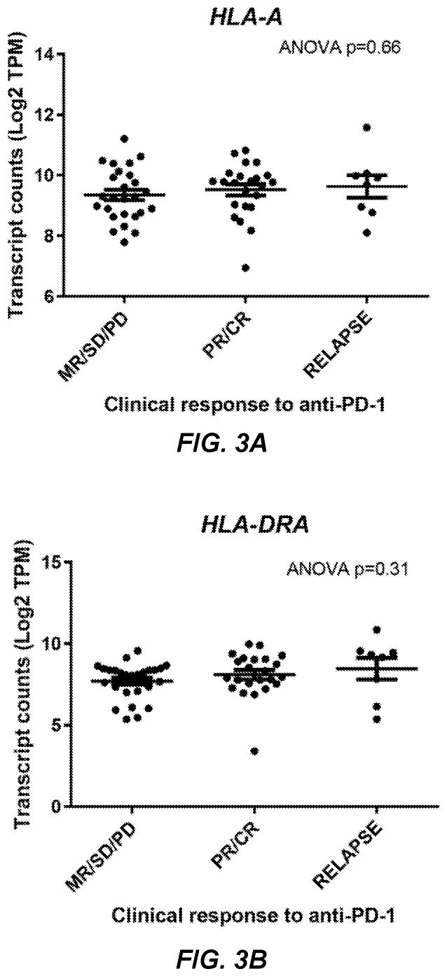

Next, the association of expression of checkpoint molecules and ligands with annotated clinical response to anti-PD-1 in these patients was examined. Included in this analysis were tumors (n=6 patients and n=8 samples, with three isolated resections/biopsies from a single patient) from patients who initially responded to anti-PD-1 therapy but subsequently progressed, where the progression sample was available for analysis. When comparing treatment response groups, LAG3 and HAVCR2 (encoding Tim-3) expression showed differential expression on analysis of variance (ANOVA). Of interest, both LAG3 and HAVCR2 expression was significantly higher in progression (relapse) specimens (i.e. acquired resistance) versus primary resistance (progressive disease/PD) (FIG. 1F). IFNG expression was also higher in relapsed patients, while an IFN-γ-response signature was not elevated but showed a similar trend, indicating that the majority of these tumors had not lost IFN-γ activity or expression (FIG. 2). Neither transcript expression of MHC-I (HLA-A) nor MHC-II (HLA-DRA) were associated with clinical response to PD-1 in patients, stressing the unique information gained by examining MHC-II protein expression by IHC specifically in tumor cells (FIG. 3). Finally, the cohort included 3 patients in whom paired tumor samples were available both prior to response to anti-PD-1, and at relapse/progression. It was hypothesized that alternative checkpoints (LAG-3 and HAVCR2) would be upregulated in matched samples after progression on PD-1. When considering checkpoint molecule expression (PDCD1, LAG3, and HAVCR2) in these three matched pairs, both LAG3 and HAVCR2 demonstrated a trend toward enrichment in 3/3 specimens (one-tailed T test p=0.15), although enrichment of LAG3 was more striking (one-tailed T test p=0.055; FIG. 1G). IHC analysis for LAG-3+ tumor-infiltrating lymphocytes (TILs) confirmed this trend (n=5 pairs, before and at acquired resistance, all unique patients; FIG. 1H-I). Increased LAG3 and HAVCR2 upon resistance to anti-PD-1 have been observed in both humanized murine models and patients.

Association of MHC-H Expression with Inflammation and LAG-3 Expression in Breast Cancer.

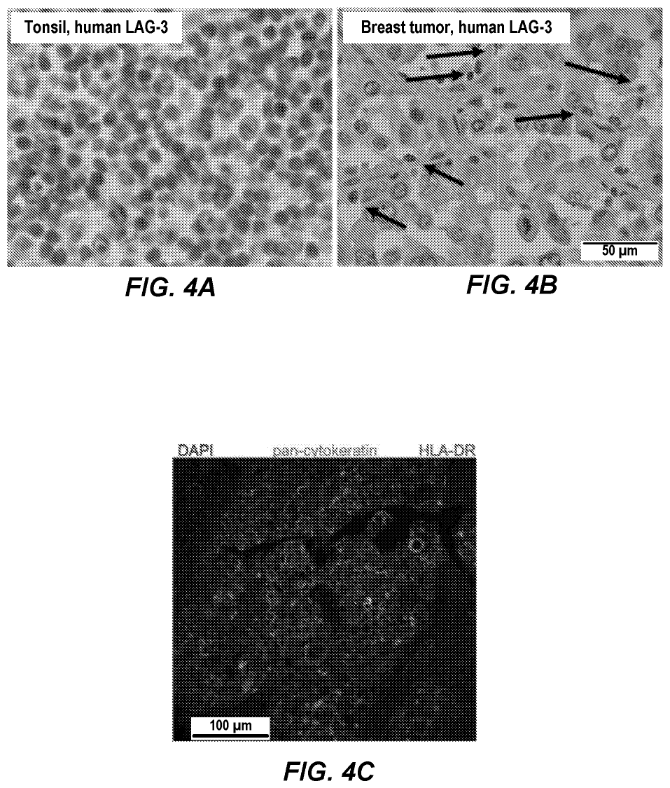

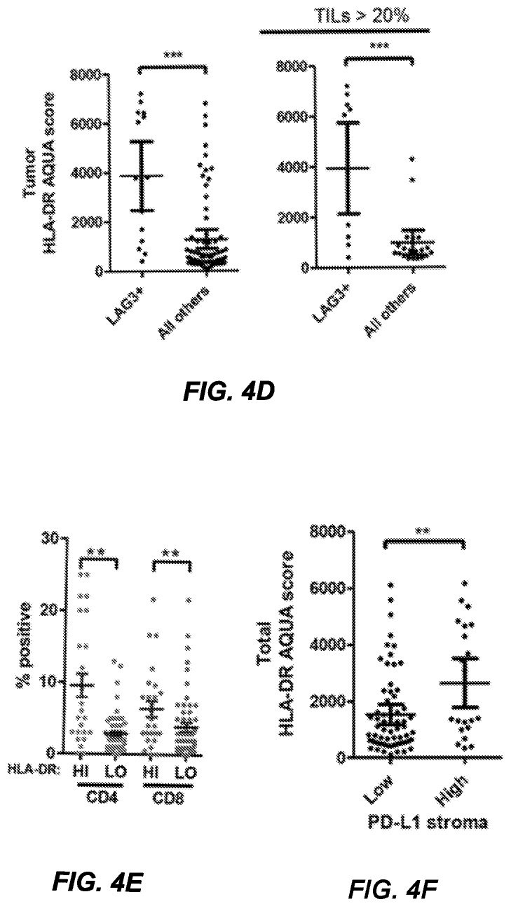

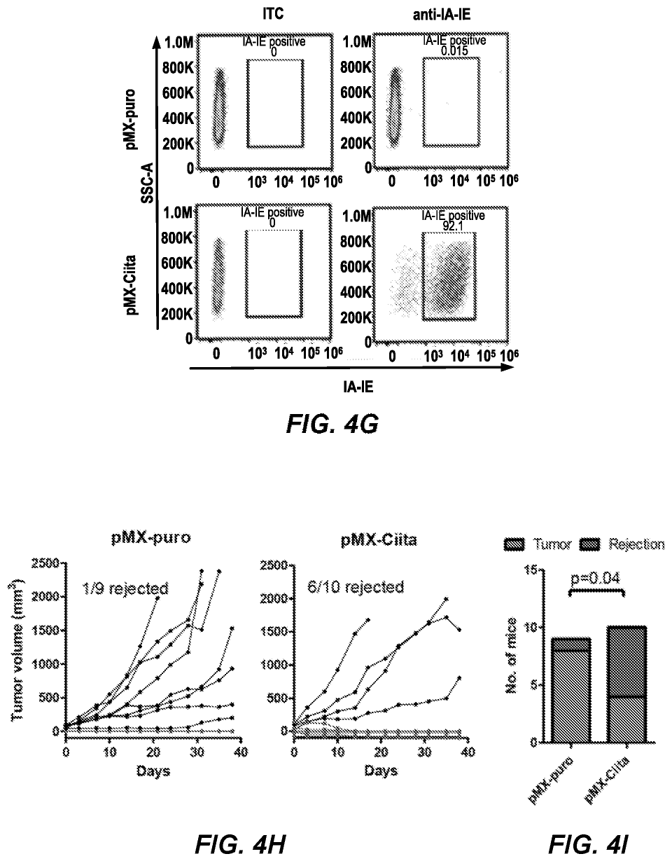

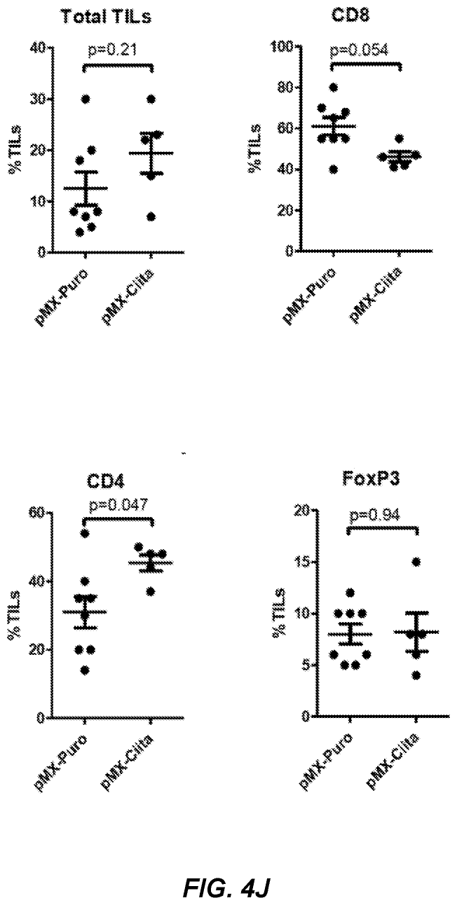

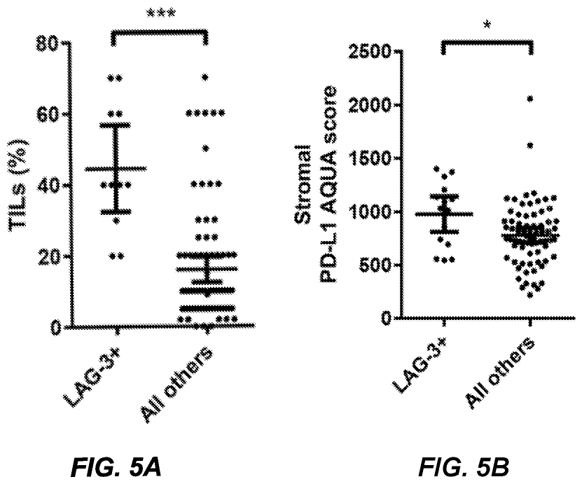

Tumor cell-autonomous MHC-II expression is an important biomarker in breast cancer. MHC-II+ breast tumors were found to have a greater degree of TILs after neoadjuvant chemotherapy, which correlates with improved outcomes after surgical resection. Since this series of 112 triple-negative breast cancers (TNBC) were previously characterized for MHC-II/HLA-DR expression in the tumor compartment (Loi et al.), whether a similar association of MHC-II+ tumors with LAG-3+ TILs (FIG. 4A-B) could be observed in this cohort was investigated. HLA-DR was scored using automated quantitative analysis (AQUA; FIG. 4C). A strong association of the presence of LAG-3+ TILs in tumors with high HLA-DR expression was found, both across the entire series, and after controlling for the intrinsically higher rate of TILs in HLA-DR+ tumors by including only heavily-infiltrated (TILs>20%) tumors in the analysis (FIG. 4D and FIG. 5). Consistent with findings in melanoma (Johnson et al.), HLA-DR+ tumors were also strongly associated with the presence of CD4 and, to a lesser degree, CD8 infiltrate (FIG. 4E). HLA-DR positivity was also associated with higher PD-L1 expression in the tumor-associated stroma, but the association of PD-L1+ stroma with LAG-3+ TILs was weaker, indicating that HLA-DR positivity is associated with LAG-3+ TILs and higher PD-L1 expression in the tumor microenvironment, but that LAG-3 positivity and PD-L1 positivity are not necessarily directly correlated (FIG. 4F and FIG. 5b).

### Enforced Expression of MHC-H Promotes Tumor Rejection, CD4+ T Cell Recruitment, and a Specific Pathway to Immune Evasion.

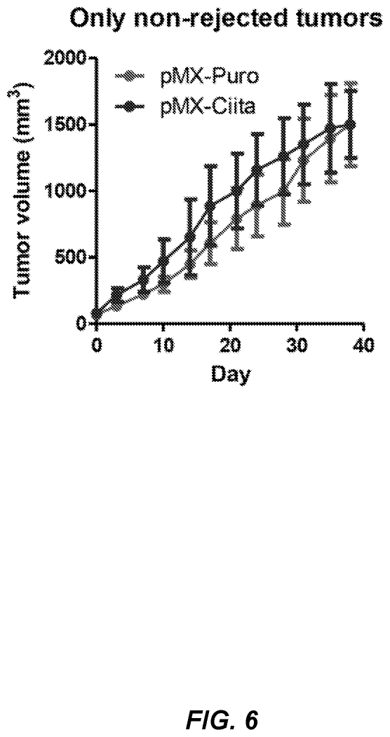

To better understand the direct role of MHC-II expression on the tumor microenvironment, expression of MHC-II on MMTV-neu breast tumor cells was enforced through transduction of Ciita, the master regulator of MHC-II. Cells transduced with Ciita were strongly IA-IE+(murine MHC-II) by flow cytometry analysis (FIG. 4G). When equivalent CIITA+ or vector control cells were injected orthotopically into the mammary fatpad of wildtype FVB/n mice, a substantially greater rejection rate was observed (FIG. 4H-I). Rejecting mice were 100% resistant to rechallenge. In mice that did form tumors, the tumor growth rate was similar in both MHC-II+ and MHC-II− tumors (FIG. 6), indicating a robust adaptive resistance to the presence of tumor-autonomous MHC-II in a subset of tumors.

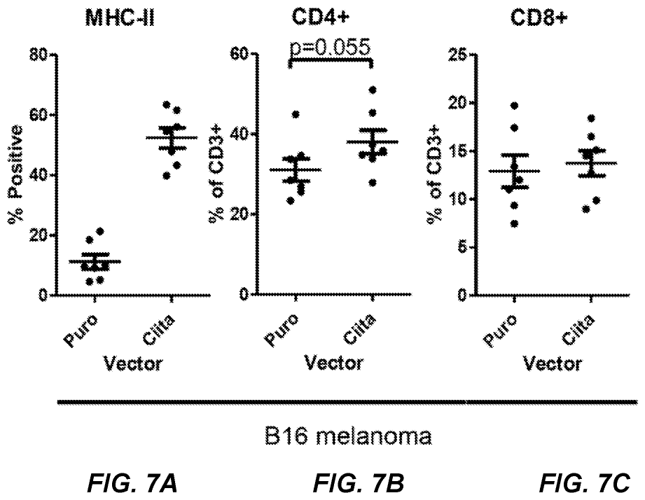

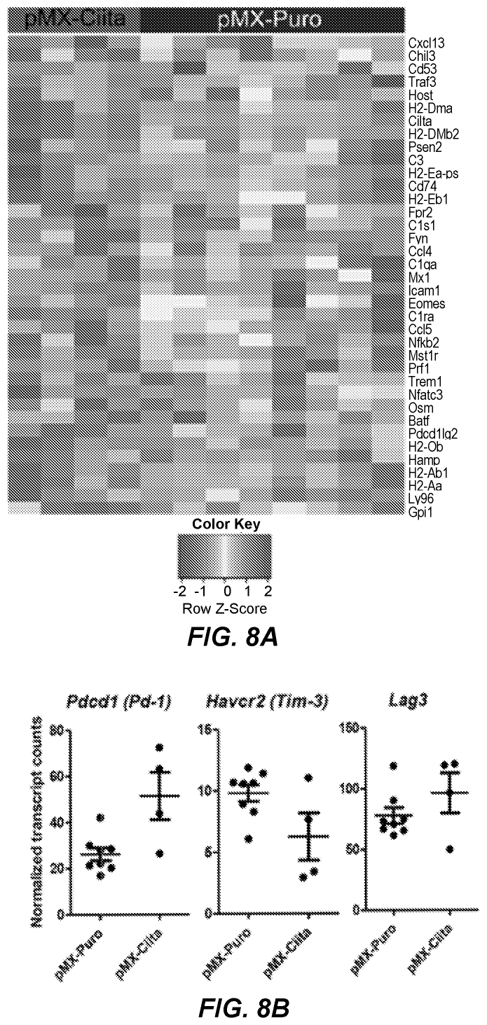

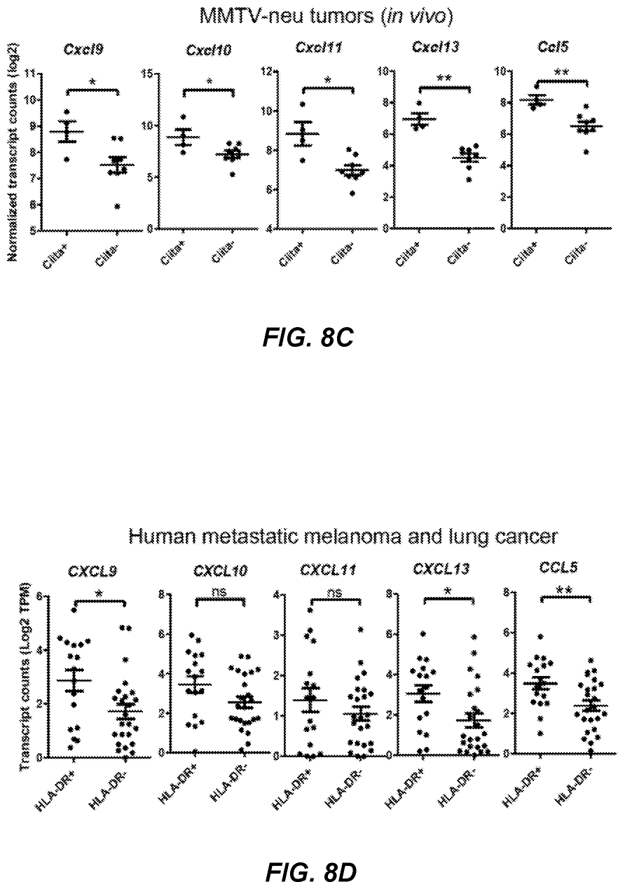

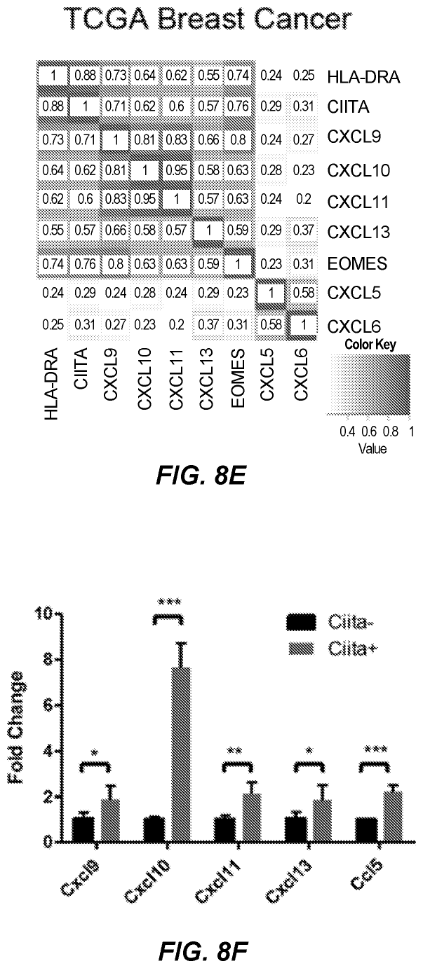

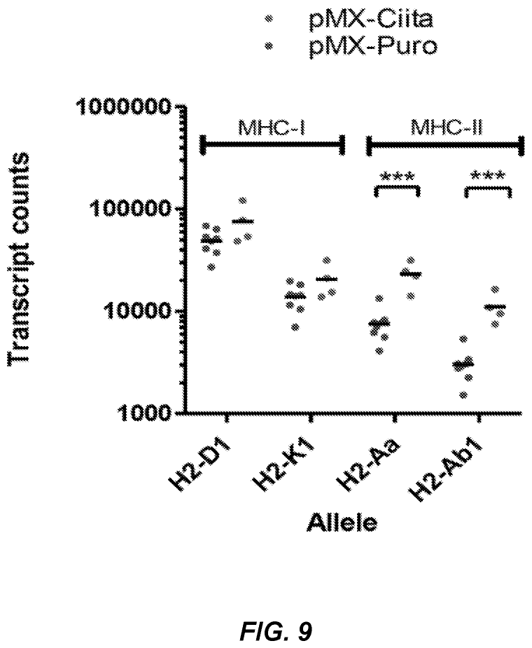

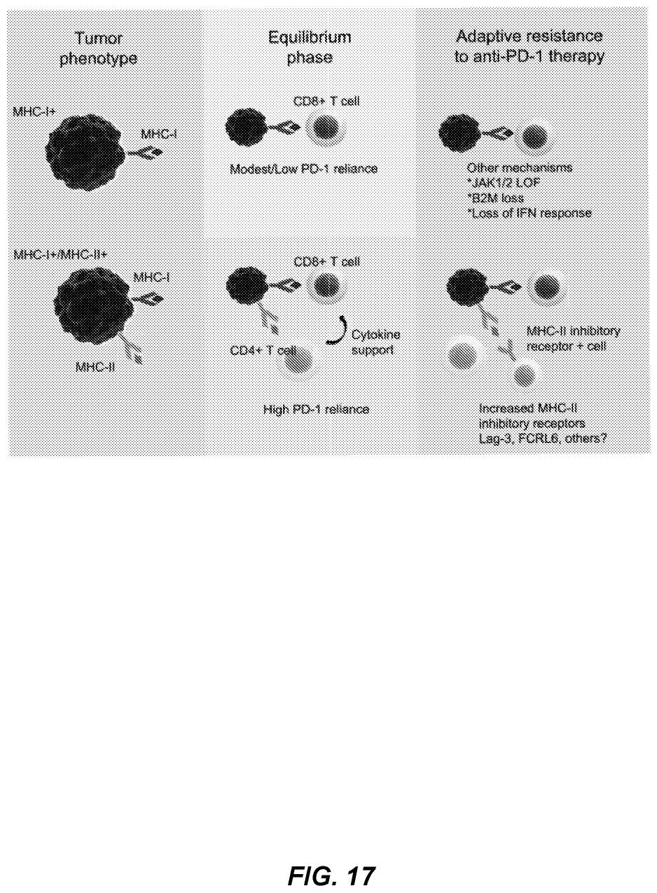

Enforced expression of MHC-II results in increased anti-tumor inflammation, Th1 differentiation, and antigen-specific CD4+ T cells. Consistent with this, tumors that did form in the presence of enforced MHC-II had greater fractions of CD4+ T cells (normalized to total TILs), with no change in the regulatory compartment (Foxp3+) (FIG. 4J). CD8+ T cells showed a downward trend when normalized to total TILs, however, this was reflective of the increased percentage of CD4+ T cells, and absolute degree of CD8+ T cell infiltrate was not affected by MHC-II status. Similar findings were observed in MHC-II-enforced B16 melanoma tumors generated in C57BL/6 mice analyzed by flow cytometry (FIG. 7), although overexpression of MHC-II in this model did not induce tumor rejection. Gene expression analysis of refractory (those tumors evading initial immunologic rejection) MHC-II+ and control MMTV-neu tumors showed enhanced expression Ciita-regulated genes (indicating MHC-II expression was not lost in these tumors) (FIG. 8 and FIG. 9). In addition, Both Pdcdl/Pd-1 and Lag3 were more highly expressed in MHC-II+ tumors. Havcr2/Tim-3 was not highly expressed and not enriched in MHC-II+ tumors (FIG. 8B). Collectively, these data show that MHC-II expression in tumor cells promotes an anti-tumor immune environment coinciding with recruitment of CD4+ T cells, and the eventual engagement of Pd-1 and Lag-3 to suppress anti-tumor immunity (FIG. 17).

### MHC-H Expression Promotes the Expression of T Cell Recruiting Chemokines.

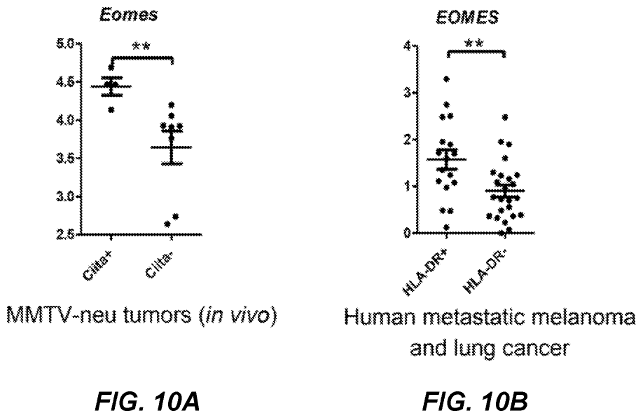

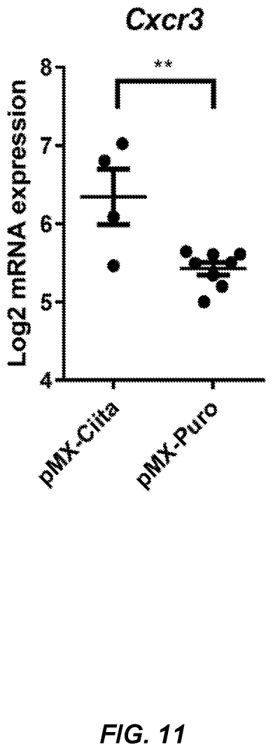

The mechanism behind recruitment of T cells to the immune microenvironment of MHC-II+ tumors is unclear. However, we noticed from our gene expression analyses that known T cell chemo-attractant cytokines (Cxcl9, 10, 11, 13; Ccl5) were elevated in MHC-II+ MMTV-neu tumors (FIG. 8C). However, Eomesodermin (Eomes) expression was elevated as well in these tumors, indicating the potential for effector T cell exhaustion (FIG. 10A). Similar elevation of these markers was present in MHC-II+ melanomas and lung cancers (FIG. 8D and FIG. 10). A high degree of co-expression was also observed in primary breast cancers supporting the interdependency of these markers in the MHC-II+ phenotype (FIG. 8E). Cxcl9, Cxcl10, and Cxcl11 (and their human orthologues) which are chiefly produced by tissue resident and endothelial cells, bind the Cxcr3 receptor on Th1 and cytotoxic T cells to license entry into sites of inflammation. Interestingly, increased Cxcr3 expression was also detectable in the tumor microenvironment of Ciita+ MMTV-neu tumors (FIG. 11).

To determine if chemokine production was a direct byproduct of Ciita expression, MMTV-neu cells grown ex vivo for expression of Cxcl9, 10, 11, 13 and CcI5 were evaluated. Enhanced expression of these chemokines at the mRNA level was detected, indicating they may be downstream of Ciita transcriptional activation (FIG. 8F). Consistent with this hypothesis, large-scale ChIP-seq experiments have identified DNA binding of the CIITA/RFX5 complex at the promoter of CXCL9 and CXCL10. Thus, the likely mechanism whereby CIITA expression mediates an enhanced inflammatory environment is via increased chemo-attractant cytokines which promote the recruitment of Th1 and cytotoxic T lymphocytes to the microenvironment, exacerbating inflammatory signals.

### A Combination of PD-1 and Lag-3 Immune Checkpoint Inhibitor Therapy Enhances Anti-Tumor Immunity in MHC-II+ Tumors.

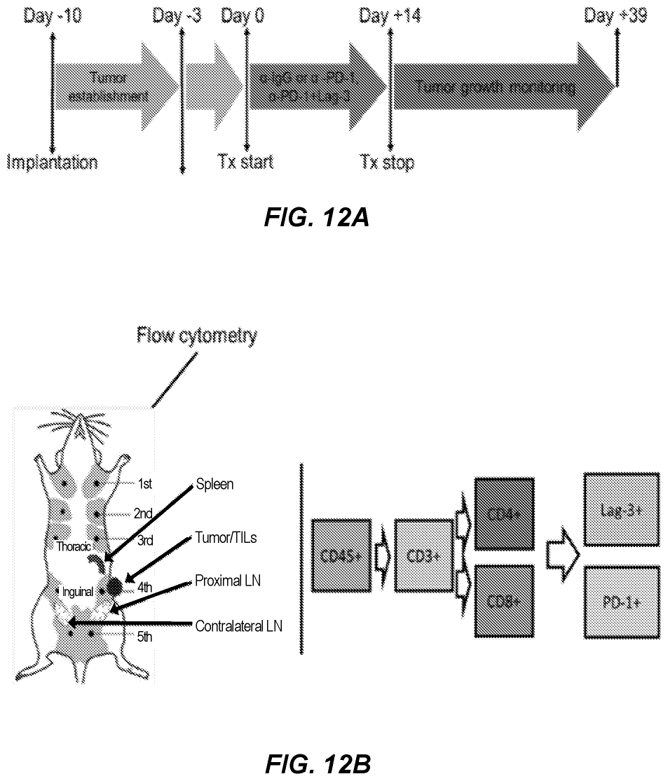

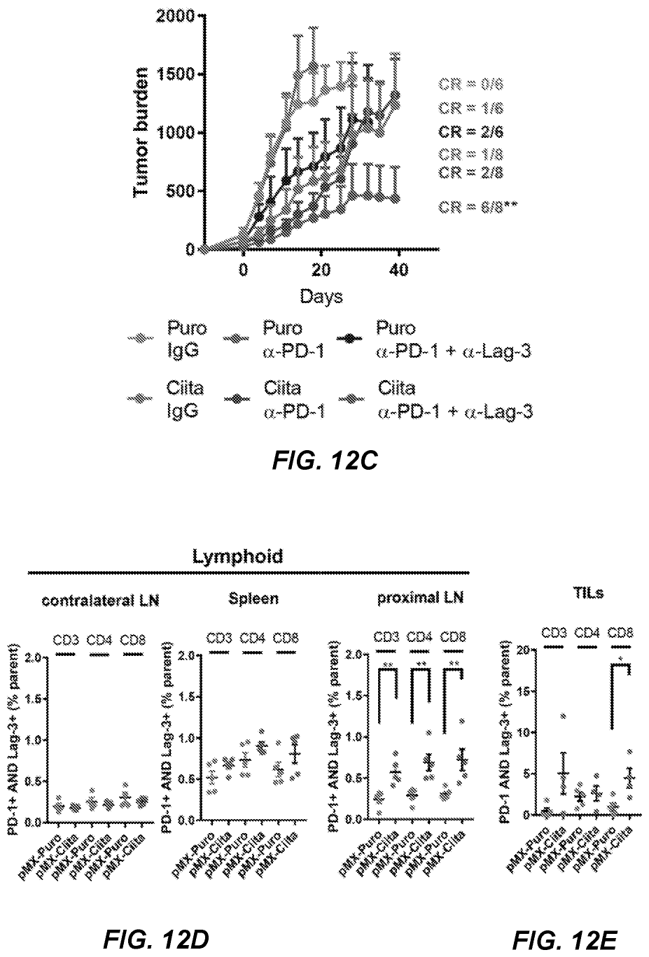

Next, it was hypothesized that MHC-II+ tumors may generate direct dependence on PD-1 and Lag-3 in T cells, resulting in the generation of tumor tolerance to maintain immunologic equilibrium. To test this hypothesis, MMTV-neu tumor cells transduced to enforce expression of Ciita (or vector control) were utilized, as above, according to the experimental design shown in FIG. 12A-B. Ten days after orthotopic implantation, mice were treated with anti-IgG (control), anti-PD-1, or the combination of anti-PD-1 and anti-Lag-3 for 2 weeks. Only mice with palpable and actively growing tumors at the start of therapy were treated in order to reduce confounders associated with enhanced immunogenicity and rejection observed in untreated Ciita+ tumors. A moderate treatment effect was observed in the combination arm in pMX-puro (control) tumors, but a pronounced anti-tumor effect was observed in the combination arm for Ciita+ tumors, with 6/8 mice exhibiting complete tumor rejection (p<0.05, χ² test); FIG. 12C). Flow cytometry analysis of lymphoid compartments demonstrated enhanced PD-1+/Lag-3+CD4+ and CD8+ T cells in the proximal lymph node, with a similar trend observed in the spleen, but not the contralateral lymph node (FIG. 12D). A similar effect was observed in tumor-infiltrating CD8 cells (FIG. 12E). Thus, MHC-II positivity on tumor cells can elicit enhanced dependency on T cell checkpoints, including Lag-3, which can be overcome therapeutically.

### Alternative MHC-II Ligands are Upregulated in MHC-II+ Tumors and Promote Suppression of Effector Cell Cytotoxicity.

Given these findings, it was hypothesized that other MHC-II receptors on lymphocytes may exist with similar functionality to Lag-3. FCRL6 is an ITIM-bearing Ig superfamily member expressed by cytotoxic NK cells and effector memory CD8+ T cells that interacts with MHC II and MHC I. Thus, it was hypothesized that FCRL6 may function as a novel immune checkpoint to suppress effector cell activity when engaged with MHC-II.

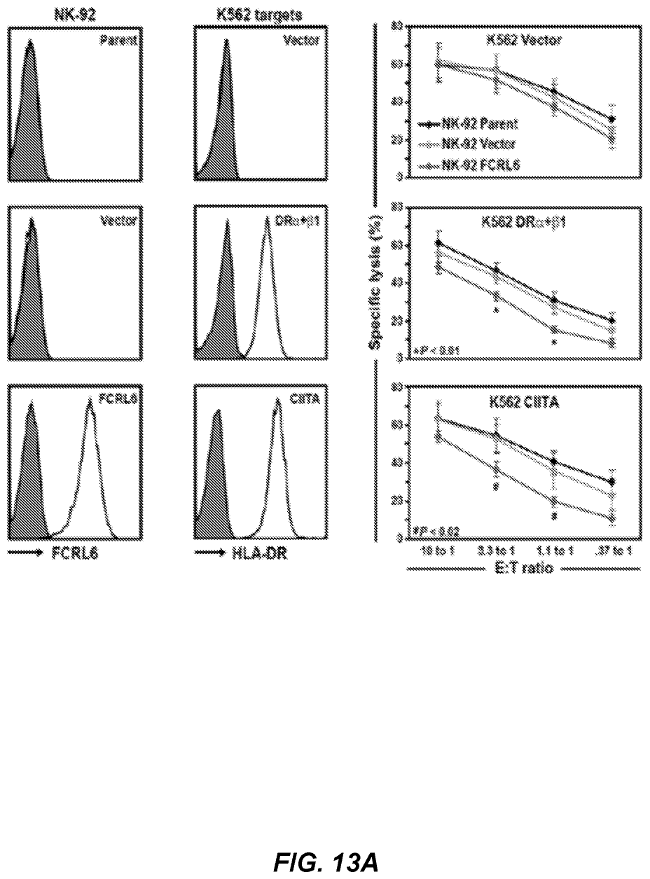

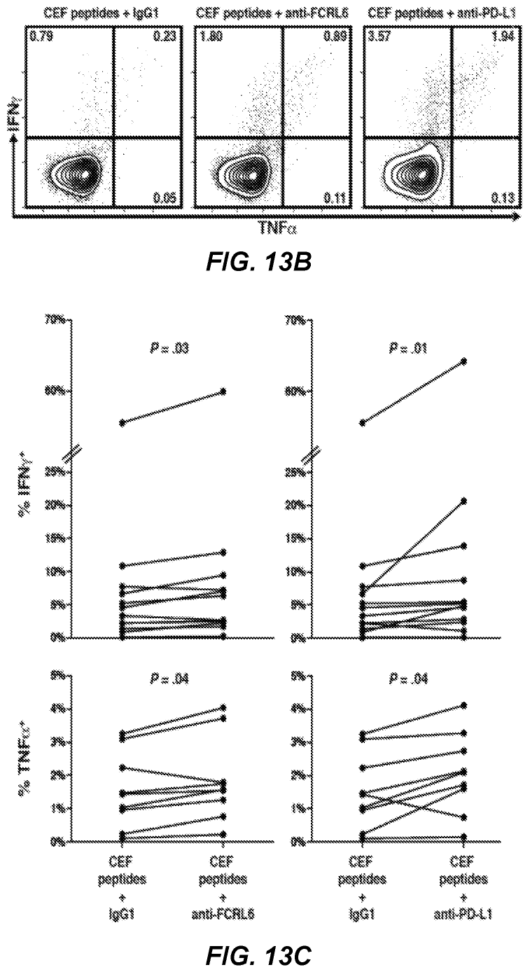

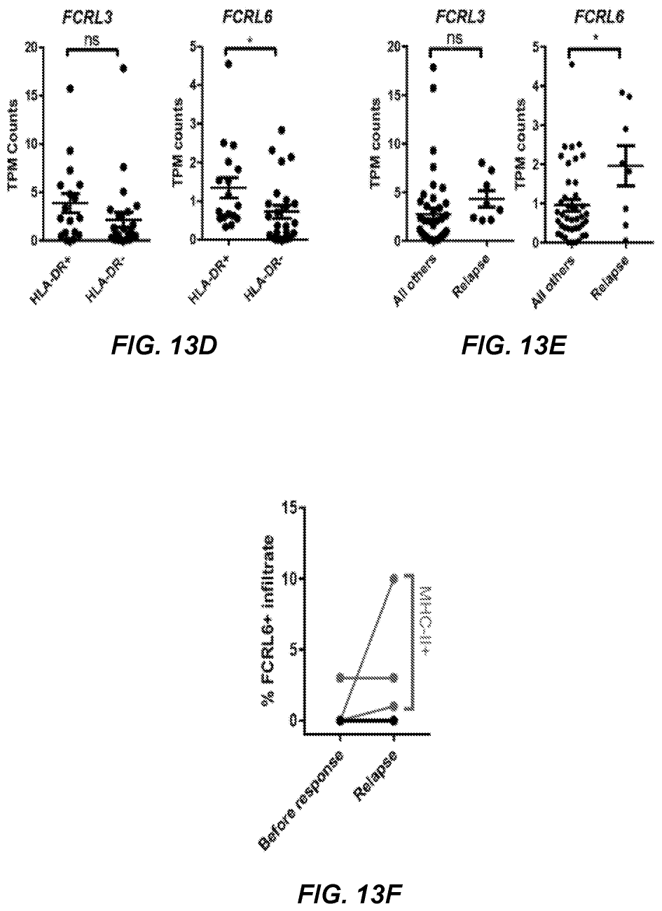

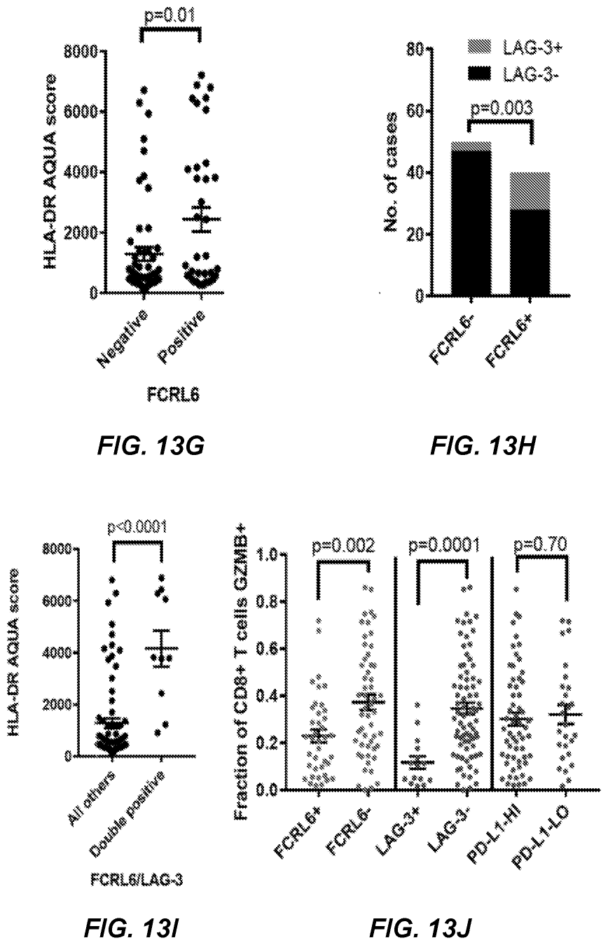

In order to determine whether FCRL6 inhibits NK cell-mediated killing of HLA-DR-expressing tumor cells, the NK-92 human cytotoxic NK cell line, which does not endogenously express FCRL6 on its surface, was transduced with either FCRL6 or a vector control (FIG. 13A). In addition, K562 target cells were transduced either with plasmids harboring the HLA-DRα and -DR1 subunits or with CITA to drive endogenous MHC class II expression in these cells. K562 HLA-DRα+β1 and CIITA transductants, but not parental K562 cells or control transductants, expressed surface HLA-DR by flow cytometric analysis (FIG. 13A). K562 cells lack endogenous MHC-I expression, and are therefore targeted by NK cell-mediated cytotoxicity. Thus, FCRL6+NK-92 lines were assayed for their ability to kill K562 transductants (MHC-II+ and negative) in ⁵¹Cr release experiments. These studies found that FCRL6+NK-92 cells were significantly impaired in their capacity to lyse K562 DRα+β1- or CIITA-expressing targets but not control K562 cells (FIG. 13A). Hence, FCRL6 is an inhibitory NK receptor for HLA-DR. Due to lack of a functional homolog in mice, only human systems are equipped to demonstrate this. Since mouse FCRL6 differs structurally and functionally from its human relative, it is not a viable interspecies translational model for study. To test the hypothesis that the FCRL6/HLA-DR interaction might also inhibit CD8+ T cell responses, the effect of FCRL6 blockade during pathogen-specific peptide stimulation in vitro was examined. This analysis employed an anti-FCRL6 mAb (1D8) that is capable of blocking FCRL6 activation in co-culture assays and obstructing FCRL6-Fc binding to HLA-DR transductants (Schreeder et al., “Cutting edge: FcR-like 6 is an MHC class II receptor. J Immunol 185, 23-27 (2010)).

Blood mononuclear cells from healthy donors were stimulated with pooled antigenic MHC class I-restricted peptides from CMV, EBV, and influenza virus epitopes (CEF peptide pool) in the presence of anti-FCRL6 or anti-PD-L1 (a positive control). Following a 6-day culture period, cells were collected, re-stimulated with CEF, and assayed for cytokine production by intracellular staining. A significantly higher frequency of CD8+ T cells produced IFNγ and TNFα when cultured with the FCRL6 or PD-L1 mABs compared to controls (FIG. 13B-C). Thus, these experiments identify the MHC-II receptor FCRL6 as a potential immunotherapeutic target and suppressor of NK and effector T cell activity.

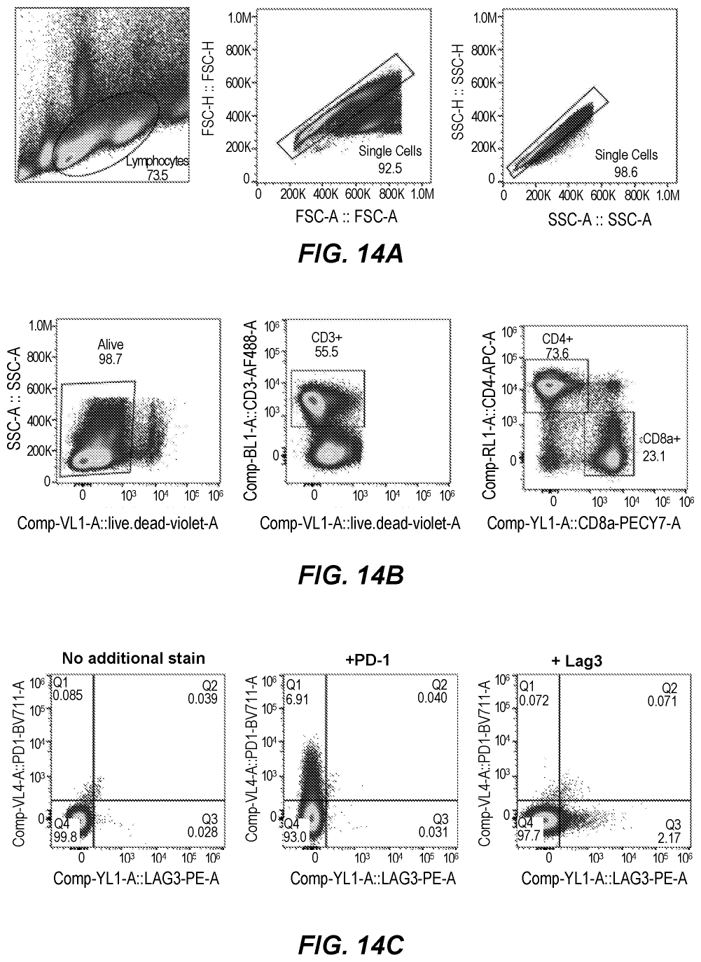

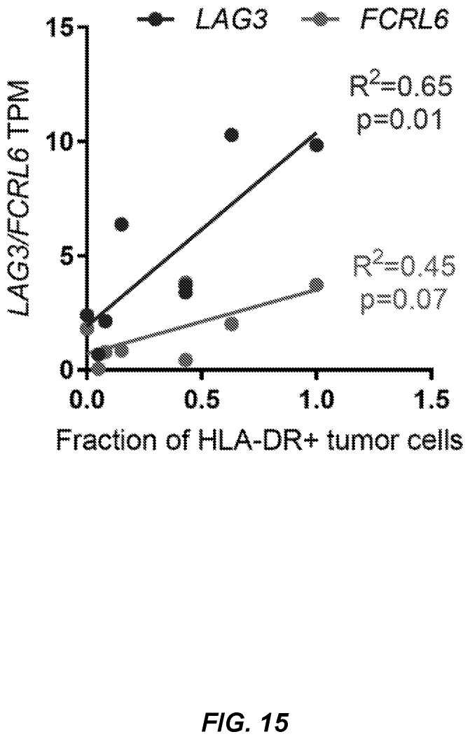

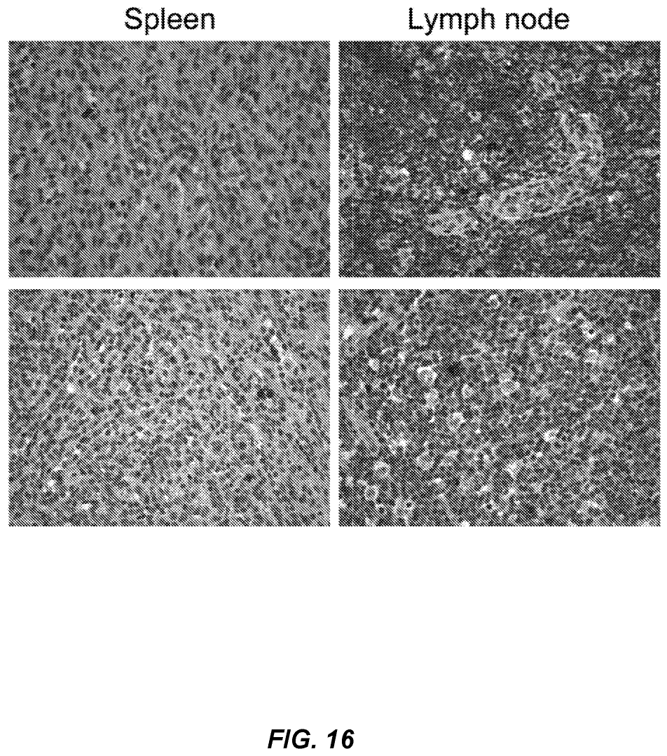

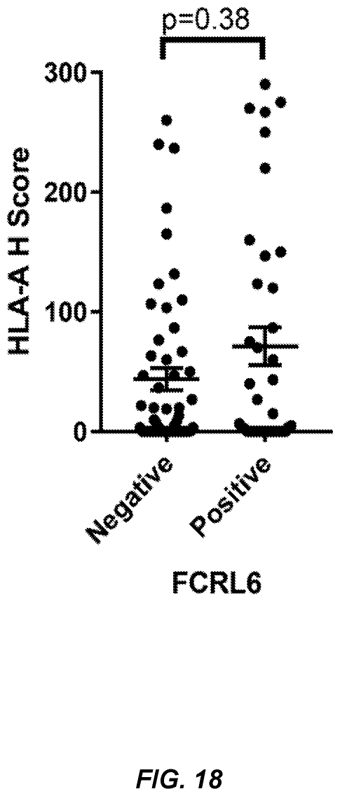

Surprisingly, it was found that FCRL6, but not its relative FCRL3, was significantly more highly expressed in MHC-II melanoma and lung cancers (FIG. 13D) and the degree of LAG3 and FCRL6 showed a linear relationship with the fraction of HLA-DR+ tumor cells (R²=0.65 and 0.45, respectively; FIG. 14). Furthermore, FCRL6 was elevated at relapse after progression on PD-1-targeted therapy both by mRNA (FIG. 13E) and at the protein level (FIG. 13F, FIG. 15). In triple-negative breast cancer specimens, FCRL6+ lymphocytes were associated with tumor HLA-DR status (FIG. 13G) and Lag-3 status (FIG. 13H), but not MHC-I status (HLA-A; FIG. 16, FIG. 18). Furthermore, concurrent presence of Lag-3+ lymphocytes and FCRL6+ lymphocytes was strikingly associated with high tumor-specific HLA-DR expression (FIG. 13I). Finally, among all breast tumors assessed, the presence of FCRL6 or Lag-3+ lymphocytes was associated with fewer cytotoxic CD8+ T cells (as defined by granzyme-B+/CD8+ cells), indicating active immune suppression mediated by these checkpoints (FIG. 13J), particularly in MHC-II+ tumors. Collectively, these data show that like LAG-3, FCRL6 could be a novel inhibitory MHC-II receptor that is engaged preferentially in MHC-II+ tumors, and may be partially responsible for adaptive resistance to anti-PD-1 therapy (FIG. 17).

Although anti-PD-1 based therapy may produce durable anti-tumor immune responses in some patients, a complex landscape of both intrinsic and acquired therapeutic resistance is emerging. As shown herein, MHC-II expression characterizes a particularly T cell-inflamed and immune-responsive subset of solid tumors. Also, as provided herein, this tumor-autonomous phenotype drives specific, context-dependent immune-escape mechanisms involving alternative ligands to MHC-II, specifically LAG-3 and FCRL6.

These data shed new light on how tumor-specific antigen presentation via MHC-II plays a role in modifying anti-tumor immunity. The findings provided herein, demonstrating a class-II targeting mechanism of acquired resistance to PD-1 therapy preferentially occurring in MHC-II+ tumors (i.e. upregulation of the inhibitory MHC-II receptors, LAG-3 and FCRL6), provide support for the in vivo and clinical importance of tumor-specific class II expression. Finally, upregulation of inflammation-site licensing T cell chemokines in MHC-II+ tumors, downstream of CIITA activity, represents an intriguing additional mechanism whereby CIITA-mediated class II expression may recruit T cells to the tumor microenvironment.

Adaptive resistance to anti-PD-1 is an increasingly recognized clinical problem. While some responses appear remarkably durable, >50% of responding patients ultimately experience disease progression. The clinical management of these patients remains problematic, although some individuals may experience excellent clinical outcomes with anti-PD-1 re-induction or management of isolated relapses. Effective combinatorial strategies are thus urgently needed, and the data provided herein reveal specific therapeutic approaches for MHC-II+ tumors.

Finally, these data indicate that molecular profiling at resistance may uncover therapeutic strategies uniquely suited for particular tumor subsets. This could provide a more rational approach than the current landscape of clinical trials that tests anti-PD-1 based combinations in patients who progress following monotherapy. In particular, PD-1/PD-L1 and LAG-3 blockade could provide particular benefit for MHC-II expressing tumors.

In conclusion, the data provided herein suggest that MHC-II positivity on tumor cells provides selective pressure for LAG-3+ and FCRL6+ TILs to suppress MHC-II-mediated antigen presentation. Thus, the MHC-II+ phenotype may direct a specific pathway of adaptive resistance to PD-1-targeted immunotherapy. Further, MHC-II+ tumor expression could enrich for cancer patients likely to respond to a combination of PD-1 and LAG-3 blockade or a combination of PD-1 and FCRL6 blockade.

1. A method of reducing resistance to an anti-programmed cell death protein 1 (PD-1) or an anti-programmed cell death protein ligand 1 (PDL-1) therapy in a subject with cancer comprising administering to the subject with cancer an effective amount of an Fc receptor like 6 (FCRL6) inhibitor, wherein the subject has been treated with an anti-PD1 therapy or an anti-PDL-1 therapy.

2. The method of claim 1, wherein the cancer is selected from the group consisting of breast cancer, melanoma, bladder cancer, lung cancer, kidney cancer, head and neck cancer and Hodgkin lymphoma.

3. The method of claim 2, wherein the breast cancer is a triple negative breast cancer.

4. (canceled)

5. (canceled)

6. (canceled)

7. The method of claim 1, wherein the treatment with the anti-PD1 therapy or the anti-PDL-1 therapy occurs prior to or concurrently with administration of the FCRL6 inhibitor.

8. The method of claim 1, further comprising administering a chemotherapeutic agent or radiation therapy to the subject.

9. (canceled)

10. A method of treating or preventing a PD-1 mediated resistant cancer in a subject that has been treated with an anti-PD1 therapy or an anti-PDL-1 therapy comprising administering to the subject an effective amount of FCRL6 inhibitor.

11. The method of claim 10, wherein the cancer is selected from the group consisting of breast cancer, melanoma, bladder cancer, lung cancer, kidney cancer, head and neck cancer and Hodgkin lymphoma.

12. The method of claim 11, wherein the breast cancer is a triple negative breast cancer.

13. (canceled)

14. (canceled)

15. (canceled)

16. The method of claim 10, wherein the treatment with the anti-PD1 therapy occurs prior to or concurrently with administration of the FCRL6 inhibitor.

17. The method of claim 10, further comprising administering a chemotherapeutic agent or radiation therapy to the subject.

18. (canceled)

19. A method of diagnosing and treating a PD-1-mediated resistant cancer in a subject that has been treated with an anti-PD1 therapy or an anti-PDL-1 therapy comprising:
a) obtaining a biological sample from a subject that has been treated with an anti-PD1 therapy or an anti-PDL-1 therapy;
b) detecting the level of FCRL6 in the sample;
c) diagnosing the subject with a PD-1 mediated resistant cancer when the level of FCRL6 in the sample is increased as compared to a control sample;
d) administering an effective amount of an FCRL6 inhibitor.

20. The method of claim 19, wherein the cancer is selected from the group consisting of breast cancer, melanoma, bladder cancer, lung cancer, kidney cancer, head and neck cancer and Hodgkin lymphoma.

21. The method of claim 20, wherein the breast cancer is a triple negative breast cancer.

22. The method of claim 19, wherein the sample is from a tumor in the subject.

23. (canceled)

24. (canceled)

25. (canceled)

26. The method of claim 19, further comprising administering an anti-PD-1 therapy to the subject.

27. The method of claim 19, further comprising administering a chemotherapeutic agent or radiation therapy to the subject.

28. (canceled)

29. A method of diagnosing and treating a PD-1-mediated resistant cancer in a subject that has been treated with an anti-PD1 therapy or an anti-PDL-1 therapy comprising:
a) obtaining a biological sample from a subject that has been treated with an anti-PD1 therapy or an anti-PDL-1 therapy;
b) detecting the level of FCRL6 and the level of LAG3 in the sample;
c) diagnosing the subject with a PD-1 mediated resistant cancer when the level of FCRL6 and the level of LAG3 in the sample are increased as compared to a control sample;
d) administering an effective amount of an anti-LAG3 therapy and/or an FCRL6 inhibitor to the subject.

30. The method of claim 29, wherein the cancer is selected from the group consisting of breast cancer, melanoma, bladder cancer, lung cancer, kidney cancer, head and neck cancer and Hodgkin lymphoma.

31. The method of claim 30, wherein the breast cancer is a triple negative breast cancer.

32. The method of claim 29, wherein the sample is from a tumor in the subject.

33. (canceled)

34. (canceled)

35. (canceled)

36. The method of claim 29, further comprising administering an anti-PD-1 therapy to the subject.

37. The method of claim 29, further comprising administering a chemotherapeutic agent or radiation therapy to the subject.

38. (canceled)
